(19) United States
(12) Patent Application Publication
(10) Pub. No.: US 2013/0323205 A1
(43) Pub. Date: Dec. 5, 2013

(54) ONCOLYTIC ADENOVIRAL VECTORS AND METHODS AND USES RELATED THERETO

(71) Applicants: Iulia Diaconu, Houston, TX (US); Sari Pesonen, Helsinki (FI); Akseli Hemminki, Helsinki (FI); Vincenzo Cerullo, Helsinki (FI)

(72) Inventors: Iulia Diaconu, Houston, TX (US); Sari Pesonen, Helsinki (FI); Akseli Hemminki, Helsinki (FI); Vincenzo Cerullo, Helsinki (FI)

(73) Assignee: ONCOS THERAPEUTICS OY, Helsinki (FI)

(21) Appl. No.: 13/825,415

(22) PCT Filed: Sep. 23, 2011
(86) PCT No.: PCT/FI2011/050826

§ 371 Date: Aug. 20, 2013

(30) Foreign Application Priority Data
Sep. 24, 2010 (FI) 20105988
May 11, 2011 (FI) 20115453

(51) Int. Cl. C12N 15/86; A61K 38/19; A61K 48/00

(52) U.S. Cl. CPC C12N 15/86; A61K 48/0058; A61K 38/191
USPC 424/93.2; 435/320.1; 435/325; 435/456

(57) ABSTRACT

The present invention relates to the fields of life sciences and medicine. Specifically, the invention relates to cancer therapies. More specifically, the present invention relates to oncolytic adenoviral vectors and cells and pharmaceutical compositions comprising said vectors. The present invention also relates to said vectors for treating cancer in a subject and a method of treating cancer in a subject. Furthermore, the present invention relates to methods of producing CD40L in a cell and increasing tumor specific immune response and apoptosis in a subject, as well to uses of the oncolytic adenoviral vector of the invention for producing CD40L in a cell and increasing tumor specific immune response and apoptosis, while decreasing tumor-associated immunosuppression, in a subject.

### FIELD OF THE INVENTION

The present invention relates to the fields of life sciences and medicine. Specifically, the invention relates to cancer therapies. More specifically, the present invention relates to oncolytic adenoviral vectors and cells and pharmaceutical compositions comprising said vectors. The present invention also relates to said vectors for treating cancer in a subject and a method of treating cancer in a subject. Furthermore, the present invention relates to methods of producing CD40L in a cell and increasing tumor specific immune response and apoptosis in a subject, as well as to uses of the oncolytic adenoviral vectors for producing CD40L in a cell and increasing tumor specific immune response and apoptosis in a subject.

### BACKGROUND OF THE INVENTION

Cancer can be treated with surgery, hormonal therapies, chemotherapies, radiotherapies and/or other therapies but in many cases, cancers, which often are characterized by an advanced stage, cannot be cured with present therapeutics. Therefore, novel cancer cell targeted approaches, such as gene therapies, are needed.

During the last twenty years gene transfer technology has been under intensive examination. The aim of cancer gene therapies is to introduce a therapeutic gene into a tumor cell. These therapeutic genes introduced to a target cell may, for example, correct mutated genes, suppress active oncogenes or generate additional properties to the cell. Suitable exogenous therapeutic genes include but are not limited to immunotherapeutic, anti-angiogenic, chemoprotective and “suicide” genes, and they can be introduced to a cell by utilizing modified virus vectors or non-viral methods including electroporation, gene gun and lipid or polymer coatings.

Requirements of optimal viral vectors include an efficient capability to find specific target cells and express the viral genome in the target cells. Furthermore, optimal vectors have to stay active in the target tissues or cells. All these properties of viral vectors have been developed during the last decades and, for example retroviral, adenoviral and adeno-associated viral vectors have been widely studied in biomedicine.

To further improve tumor penetration and local amplification of the anti-tumor effect, selectively oncolytic agents, e.g., conditionally replicating adenoviruses, have been constructed. Oncolytic adenoviruses are a promising tool for treatment of cancers and have shown good safety and some efficacy in clinical trials. Tumor cells are killed by oncolytic adenoviruses due to the replication of the virus in a tumor cell, the last phase of the replication resulting in a release of thousands of virions into the surrounding tumor tissues for effective tumor penetration and vascular re-infection. Due to engineered changes in the virus genome, which prevent replication in non-tumor cells, tumor cells allow replication of the virus while normal cells are spared.

Replication can be limited to the tumor tissue either by making partial deletions in the adenoviral E1 region or by using tissue or tumor specific promoters (TSP). Insertion of such a promoter may enhance effects of vectors in target cells and the use of exogenous tissue or tumor-specific promoters is common in recombinant adenoviral vectors.

Previous studies have shown that the human telomerase reverse transcriptase (hTERT) promoter is highly active in most tumor and immortal cell lines but inactive in normal somatic cell types. hTERT is the catalytic subunit of telomerase and functions to stabilize telomere length during chromosomal replication. Oncolytic adenoviruses utilizing the hTERT promoter to control adenoviral early region genes have been previously described (see, for instance Huang, T G, et al., Gene Therapy 2003; 10, 1241-1247; Ryan, PC. et al., Cancer Gene Therapy 2004; 11, 555-559; Irving et al., Cancer Gene Therapy 2004; 11, 174-185; Bauerschmitz G J, et al., Cancer Res 2008; 68: 5533-9). However, telomerase is expressed, besides in tumor cells, also in other human cells with an unlimited proliferative potency, such as stem cells.

Most clinical trials have been performed with early generation adenoviruses based on adenovirus 5 (Ad5). The anti-tumor effect of oncolytic adenoviruses depends on their capacity for gene delivery. Unfortunately, most tumors have low expression of the main Ad5 receptor, wherefore modifications have been introduced to the Ad5 capsid. For instance, a capsid modification with the serotype 3 knob has shown improved infectivity and good efficacy in ovarian cancer (Kanerva A, et al., Clin Cancer Res 2002; 8:275-80; Kanerva A, et al., Mol Ther 2002; 5:695-704; Kanerva A, et al., Mol Ther 2003; 8:449-58). Also, as the fiber and the penton base of the Ad vectors are key mediators of the cell entry mechanism, targeting of recombinant Ad vectors may be achieved via genetic modifications of these capsid proteins (Dmitriev I., et al. 1998, Journal of Virology, 72, 9706-9713). Currently most oncolytic viruses in clinical use are highly attenuated in terms of replication due to several deletions in critical viral genes. These viruses have shown excellent safety record, but the antitumor efficacy has been limited.

However, clinical and preclinical results show that treatment with unarmed oncolytic viruses is not immunostimulatory enough to result in sustained anti-tumoral therapeutic immune responses. In this regard, oncolytic viruses have been armed to be more immunostimulatory. Moreover, viral replication and expression of immunomodulatory proteins within a tumor potentiates the immune system by inducing cytokine production and release of tumor antigens (Ries S J, et al., Nat Med 2000; 6:1128-33).

Arming oncolytic viruses combines the advantages of conventional gene delivery with the potency of replication competent agents. One goal of arming viruses is the induction of an immune reaction towards the cells that allow virus replication. As mentioned above, virus replication alone, although immunogenic, is normally not enough to induce effective anti-tumor immunity. To strengthen the induction of therapeutic immunity, viruses have been armed with stimulatory proteins, such as cytokines, for the facilitation of the introduction of tumor antigens to antigen presenting cells, such as dendritic cells, and their stimulation and/or maturation. Introduction of immunotherapeutic genes into tumor cells and, furthermore, their translation of the proteins, leads to the activation of the immune response and to more efficient destruction of tumor cells. The most relevant immune cells in this regard are natural killer cells (NK) and cytotoxic CD8+ T-cells.

CD40 ligand (CD40L) is a type II transmembrane protein belonging to the tumor necrosis factor family. CD40L is also known as CD154 or gp39 and is predominately expressed on CD4⁺ T-cells and binds to the CD40 receptor on the membrane of antigen-presenting cells (APCs) (Grewal I S and Flavell R A., Ann Rev Immunol 1998; 16:111-35; Roy M, et al., J Immunol 1993; 151:2497-510). CD40 is expressed on macrophages and dendritic cells (DCs) where its activation by CD40L leads to antigen presentation and cytokine production followed by T-cell priming and a strong innate immune response (van Kooten C and Banchereau J., J Leukoc Biol 2000; 67:2-17). Interactions between CD40L and its receptor CD40 provide critical co-stimulatory signals that trigger T-lymphocyte expansion (Grewal I S and Flavell R A, 1998, Annu Rev Immunol 1998; 16:111-35), and increase IL-12 production which is required for the engagement of cytotoxic T lymphocytes (CTL) in the anti-tumor immune response (Loskog A S, et al., Clin Cancer Res 2005; 11:8816-21; Mackey M F, et al., J Immunol 1998; 161:2094-8). Previous observations demonstrate that recombinant soluble protein CD40L (rsCD40L) has direct effects in suppression of the tumor cell proliferation in vitro (Eliopoulos A G, et al., Oncogene 1996; 13:2243-54; Tong A W, et al., Clin Cancer Res 2001; 7:691-703) and in vivo (Eliopoulos A G, et al., Mol Cell Biol 2000; 20:5503-15; Hirano A, et al., Blood 1999; 93:2999-3007). Other direct effects of rsCD40L are the stimulation of survival signaling pathways (including PI-3-kinase and ERK/MAPK) and the induction of apoptosis in carcinoma cells (Eliopoulos, A G., et al., Mol Cell Biol 2000; 20:5503-15; Davies C C, et al., J Biol Chem 2004; 279:1010-921).

Some recent reports have shown that adenoviruses armed with CD40L can induce inhibition of tumor growth together with an increase of apoptotic events at the tumor site (Loskog A S, et al., Clin Cancer Res 2005; 11:8816-21; Fernandes M S, et al., Clin Cancer Res 2009; 15:4847-56; Loskog A S, et al., J Immunol 2004; 172:7200-5). Also, there is some evidence with regard to the anti-tumor immune response through an increased lymphocyte infiltration and the presence of cytotoxic T-cells-CD8⁺ (Hanyu K, et al., Anticancer Res 2008; 28:2785-9; lida T, et al., Cancer Sci 2008; 99:2097-10324,25).

Adenoviruses are medium-sized (90-100 nm), non-enveloped icosahedral viruses, which have double stranded linear DNA of about 36 000 base pairs in a protein capsid. The viral capsid has fiber structures, which participate in attachment of the virus to the target cell. First, the knob domain of the fiber protein binds to the receptor of the target cell (e.g. CD46 or coxsackievirus adenovirus receptor (CAR)), secondly, the virus interacts with an integrin molecule and thirdly, the virus is endocytosed into the target cell. Next, the viral genome is transported from endosomes into the nucleus and the replication machinery of the target cell is utilized also for viral purposes (Russell W. C., J General Virol 2000; 81:2573-2604).

The adenoviral genome has early (E1-E4), intermediate (IX and IVa2) and late genes (L1-L5), which are transcribed in a sequential order. Early gene products affect defense mechanisms, the cell cycle and the cellular metabolism of the host cell. Intermediate and late genes encode structural viral proteins for the production of new virions (Wu and Nemerow, Trends Microbiol 2004; 12:162-168; Russell W. C., J General Virol 2000; 81; 2573-2604; Volpers C. and Kochanek S. J Gene Med 2004; 6, suppl 1: S164-71; Kootstra N. A. and Verma I. M. Annu Rev Pharmacol Toxicol 2003; 43: 413-439).

More than 50 different serotypes of adenoviruses have been found in humans. Serotypes are classified into six subgroups A-F and different serotypes are known to be associated with different conditions, i.e., respiratory diseases, conjunctivitis and gastroenteritis. Adenovirus serotype 5 (Ad5) is known to cause respiratory diseases and it is the most common serotype studied in the field of gene therapy. In the first Ad5 vectors E1 and/or E3 regions were deleted enabling insertion of foreign DNA to the vectors (Danthinne X, Imperiale M J., Gene Therapy. 2000; 7:1707-1714). Furthermore, deletions of other regions as well as further mutations have provided extra properties to viral vectors. Indeed, various modifications of adenoviruses have been suggested for achieving efficient anti-tumor effects.

US2010047208 A1 discloses knob-modified adenovirus vectors, in which the tumor targeting is achieved with a modified hTERT promoter and which can be armed with an immunostimulatory protein, such as GM-CSF.

Still, more efficient and accurate gene transfer as well as increased specificity and sufficient tumor killing ability of gene therapies are warranted. Safety records of therapeutic vectors must also be excellent. The present invention provides a cancer therapeutic tool with these aforementioned properties by utilizing both oncolytic and immunotherapeutic properties of adenoviruses in a novel and inventive way.

### BRIEF DESCRIPTION OF THE INVENTION

The object of the invention is to provide novel methods and tools for achieving the above-mentioned properties of adenoviruses and thus, solving the problems of conventional cancer therapies. More specifically, the invention provides novel methods and tools for gene therapy.

The present application describes the construction of recombinant viral vectors, methods related to the vectors, and their use in tumor cells lines, animal models and cancer patients.

The present invention relates to an oncolytic adenoviral vector comprising

1) an adenovirus serotype 5 (Ad5) nucleic acid backbone comprising a capsid modification, preferably a capsid modification with an adenovirus serotype 3 (Ad3) knob (Ad5/3 capsid chimerism),

2) a nucleic acid sequence encoding a tumor specific human telomerase reverse transcriptase (hTERT) promotor upstream of the E1 region; and

3) a nucleic acid sequence encoding human CD40L in the place of the deleted adenoviral genes gp19k/6.7K sequence in the E3 region.

The present invention further relates to a cell comprising the oncolytic adenoviral vector of the invention.

The present invention also relates to a pharmaceutical composition comprising the adenoviral vector of the invention.

The present invention also relates to the adenoviral vector of the invention for treating cancer in a subject.

The present invention also relates to a method of treating cancer in a subject, wherein the method comprises administration of the vector or the pharmaceutical composition of the invention to a subject suffering from cancer, especially from cancer refractory to conventional chemotherapeutic and/or radiation treatments.

Furthermore, the present invention also relates to a method of producing CD40L in a cell, wherein the method comprises:

carrying a vehicle comprising an oncolytic adenoviral vector of the invention to a cell, and

expressing CD40L of said vector in the cell.

Furthermore, the present invention also relates to a method of increasing tumor specific immune response in a subject, wherein the method comprises:

carrying a vehicle comprising an oncolytic adenoviral vector of the invention to a target cell or tissue,

expressing CD40L of said vector in the cell,

increasing the amount of cytotoxic T cells and/or natural killer cells in said target cell or tissue, and

inducing Th2->Th1 switch for enhanced cytotoxic anti-tumor activity in the tumor microenvironment.

Still, the present invention also relates to a use of an oncolytic adenoviral vector of the invention for producing CD40L in a cell.

Still, the present invention relates to an oncolytic adenoviral vector of the invention for producing CD40L in a cell.

Still, the present invention also relates to a use of an oncolytic adenoviral vector of the invention for increasing tumor specific immune response in a subject.

Still, the present invention relates to an oncolytic adenoviral vector of the invention for increasing tumor specific immune response in a subject.

The present invention provides a novel tool for the treatment of cancers, especially cancers, which are refractory to or incurable by current therapeutic approaches. Also, restrictions regarding tumor types suitable for treatment remain few compared to many other treatments. In fact all solid tumors may be treated with the proposed invention. The treatment can be given intratumorally, intracavitary, intravenously and in a combination of these. The approach can give systemic efficacy despite local injection. The approach can also eradicate cells proposed as tumor initiating (“cancer stem cells”).

Besides enabling the transport of the vector to the site of interest the vector of the invention also assures the expression and persistence of the transgene. The present invention solves a problem related to therapeutic resistance of conventional treatments. Furthermore, the present invention provides tools and methods for selective treatments, without toxicity or damages in healthy tissues. Advantages of the present invention include also different and reduced side effects in comparison to other therapeutics. Importantly, the approach is synergistic with many other forms of therapy including chemotherapy and radiation therapy, and can therefore be used in combination regimens.

Induction of an immune reaction towards cells that allow replication of unarmed adenoviruses is normally not strong enough to lead to development of therapeutic tumor immunity. In order to overcome this weakness, the present invention provides armed adenoviruses with a potent inducer of anti-tumor immunity, CD40L, which moreover induces local apoptosis in the tumor tissue.

Specifically, CD40L together with non-replicative viral vectors has been shown to have synergistic potency to enhance activity of effector cells (CD8+ T-cells) by converting Th2 chemokine patterns of T cells into a Th1 type (Loskog et al 2004, J Immunol 172: 7200-5; Bendriss-Vermare et al 2005, J Leucocyte Biol 78: 954-66). Th2 promotes production of antibodies while Th1 encourages cytotoxicity and the latter may be more advantageous when attempting to target T-cells to kill tumor cells. In this patent specification, it is shown that this phenomenon is particularly potent in the context of an oncolytic adenovirus as demonstrated by preclinical and human data.

Production of CD40L by an oncolytic adenovirus is also important, because it can recruit natural killer cells to the tumor and enhance their anti-tumor activity (Nakajima et al 1998 J Immunol 161:1901-7). Further, CD40L can enhance the function of antigen presenting cells (Nakajima et al 1998 J Immunol 161:1901-7). Finally, the CD40/CD40L interaction provides powerful inhibitory signals to suppressive cells, such as regulatory T-cells, which can result in potent stimulation of anti-tumor immune reactions (Guiducci et al 2005 Eur J Immunol 35:557-67).

Additionally, the viral replication is restricted to target cells by the use of a powerful transcriptionally targeting promoter, hTERT. The tumor specific promoter hTERT is active in practically all advanced solid tumors, but it can also mediate targeting of oncolytic adenoviruses to putative cancer initiating cells, as has been shown in cancer patient pleural effusion samples (Bauerschmitz et al Cancer Res 2008 68: 5533-9). Clinical data presented here indicates no toxicity for normal tissue stem cells, since no life threatening adverse events occurred.

The present invention achieves cancer therapy, wherein tumor cells are destroyed by virion caused oncolysis combined with various different mechanisms activating human immune response, including proliferation and activation of T-cells, macrophages and dendritic cells (DC), followed by cytokine production, which in turn induces a Th1-type immune reaction for additional stimulation of cytotoxic T-cell attack on the tumor. Additionally, CD40L-induced apoptosis promotes reduction in tumor load.

Compared to adenoviral tools of the prior art, the present invention provides a more simple, more effective, inexpensive, non-toxic and/or safer tool for cancer therapy. Furthermore, helper viruses or co-administration of recombinant molecules are not needed.

The present invention provides a new generation of infectivity enhanced and highly effective adenoviruses that retain the good safety of older viruses but result in higher levels of efficacy. Importantly, the present invention describes oncolytic adenoviruses which provide immunological factors critical with regard to the efficacy of oncolytic viruses.

The novel products of the invention enable further improvements in cancer therapy.

### BRIEF DESCRIPTION OF THE FIGURES

FIG. 1 shows a schematic of Ad5/3-hTERT-E1A-hCD40L, Ad5/3-CMV-hCD40L and Ad5/3-CMV-mCD40L. Replication competent Ad5/3-hTERT-E1A-hCD40L bears a nucleic acid sequence (SEQ ID. NO:1) encoding a tumor specific human telomerase reverse transcriptase (hTERT) promoter upstream of the E1 region and gp19k/6.7K in the E3 region has been replaced with the cDNA sequence (SEQ ID NO:2) of human CD40L (FIG. 1a). Replication deficient Ad5/3-CMV-hCD40L (FIG. 1b) and Ad5/3-CMV-mCD40L (FIG. 1c) bear hCD40L and mCD40L, respectively, in place of the E1A region and the natural E1A promoter has been replaced by a CMV promoter. ADP refers to the adenovirus death protein.

FIG. 2a shows the results of a flow cytometry analysis for hCD40L expression in the 293 cell line at 24 hours post infection with 10VP/cell. FIG. 2b shows the in vivo expression of the CD40L protein in mouse serum. For analyzing hCD40L in the serum from mice treated with Ad5/3-CMV-hCD40L, blood was collected only twice (day 4 and 8) due to rapid tumor growth and animals had to be killed on day 8. FIG. 2c shows the functionality of hCD40L expressed by replication competent adenovirus Ad5/3-hTERT-E1A-hCD40L. A plasmid featuring the Nf-κB5-ELAM promoter coding for luciferase was transfected into EJ cells and the supernatant from A549 cells infected with Ad5/3-hTERT-hCD40L was added. Mock values (non-infected) were subtracted and Nf-κB activity is expressed in fold increase of luciferase expression (relative light units, RLU). Supernatants from cells infected with an oncolytic virus without CD40L (Ad5/3-hTERT-E1A) and with human recombinant CD40L (hCD40L) were used as controls. The assay was performed three times and each time was assessed in triplicates. Data are presented as mean±SEM; ***, P<0.001. FIG. 2d shows also the functionality of hCD40L. Human B-lymphocyte cell line (Ramos-Blue) stably expresses an NF-κB/AP-1-inducible SEAP reporter gene. The supernatant collected from virus-infected cells was used to stimulate Ramos-Blue cells and as a surrogate of activation of cells, the production of SEAP was measured with the QUANTI-Blue assay reagent (InvivoGen, San Diego, Calif., USA). Data are presented as mean±SEM; ***, P<0.001.

FIG. 3 shows the oncolytic potency of Ad5/3-hTERT-E1A-hCD40L in CD40 positive (EJ) or CD40 negative (A549) cell lines. To assess the oncolytic potency of the adenovirus of the invention, A549 (CD40−) (FIG. 3a) and EJ (CD40+) (FIG. 3b) cell lines were infected with Ad5/3-hTERT-E1A-hCD40L, Ad5/3-hTERT-E1A, Ad5/3-CMV-hCD40L, and Ad5/3Luc1 at doses of 0, 1, 1, 10, 100, and 1000 VP/cell, and the cell viability was measured by the MTS assay. EJ and A549 cell monolayers were infected either with Ad5/3-hTERT-E1A-hCD40L (FIG. 3c) or Ad5/3-hTERT-E1A (FIG. 3d). The assay was stopped 7 days after infection and the cell viability was measured by the MTS assay. ***, P<0.001.

FIG. 4 shows the anti-tumor efficacy of vectors Ad5/3-CMV-hCD40L and Ad5/3-hTERT-E1A-hCD40L in mice. Mice bearing subcutaneous A549 (CD40−) (FIG. 4a) or EJ (CD40+) (FIG. 4b) tumors were injected intratumorally with the replication deficient adenovirus Ad5/3-CMV-hCD40L at a dose of 10⁸ VP/tumor on three days (0, 2 and 4, n=5 mice/group) and the tumor growth was followed. This experiment shows that CD40L has anti-tumor activity in CD40+ cells. Tumors were induced in mice with either A549 (FIG. 4c) or EJ (FIG. 4d) cell lines and injected with the replication competent adenovirus Ad5/3-hTERT-E1A-hCD40L and the control virus Ad5/3-hTERT-E1A at a dose of 10⁸VP/tumor on three days (0, 2 and 4, n=5 mice/group) and tumor volumes were plotted relative to the initial size. This experiment demonstrates the oncolytic potency of Ad5/3-hTERT-E1A-hCD40L but does not take into account the immunological activity of CD40L as hCD40 is not active in mice. Data are presented as mean±SEM. *, P<0.05; **, P<0.01; ***, P<0.001.

FIG. 5 shows caspase-3 expression in CD40+ tumors. Mice bearing EJ (CD40+) tumors were injected intratumorally with Ad5/3-hTERT-E1A-hCD40L, Ad5/3-hTERT-E1A, Ad5/3-CMV-hCD40L, and Ad5/3-Luc1 (mock) three times. After 26 days, animals were killed and tumors were collected and embedded in paraffin blocks (n=5 mice/group). Immunohistochemistry for caspase-3 was performed to study the induction of apoptosis. Positive staining is shown in brown.

FIG. 6 shows that Ad5/3-CMV-mCD40L inhibits tumor growth in an immunocompetent animal model. To study the immunological effect of CD40L without confounding due to the effect of oncolysis, C57Black mice bearing subcutaneous MB49 (mouse bladder cancer cell line) tumors were injected intratumorally with the replication deficient adenovirus Ad5/3-CMV-mCD40L or the control Ad5/3-Luc1 at a dose of 3×10⁸ VP/tumor on days 0, 2 and 4 (n=6 mice/group). The tumor size was followed and plotted relative to the size on day 0. Data are presented as mean±SEM, ***, P<0.001 (FIG. 6a). FIG. 6b shows an immunohistochemical analysis of apoptosis (active caspase-3) in tumors treated with Ad5/3-CMV-mCD40L or Ad5/3-Luc1. The active caspase-3 expression is shown in brown.

FIG. 7 describes the host immune responses in a syngeneic murine model. FIG. 7a presents cytokine analysis for IL-12, IFN-γ, TNF-α and Rantes in splenocytes of mice treated with Ad5/3Luc1 (black) or Ad5/3-CMV-mCD40L (white). Splenocytes were cultured for 24, 48 or 72 hours. IL-12 indicates activation of antigen presenting cells, while the others are markers of Th1-type immune response. For immunohistological analysis, MB49 tumors were collected at 16 days after virus injection. Four μm tumor sections were stained by immunohistochemistry for different markers. FIG. 7b shows macrophage (F4/80), leukocyte (CD45) and B-lymphocyte (CD19) stainings. In FIG. 7c, tumor sections were stained for helper (CD4+) and cytotoxic (CD8+) T cells (brown).

FIG. 8 shows a pre-treatment analysis of tumor samples for prediction of the treatment efficacy. Cell killing assay (MTS-assay) was performed on fresh pretreatment malignant pleural effusion of a patient suffering from breast cancer (R73) with an oncolytic adenovirus with an Ad5-capsid and a chimeric Ad5/3 capsid (a capsid identical with Ad5/3-hTERT-E1A-hCD40L) and with a non-oncolytic adenovirus.

FIG. 9 shows the induction of adenovirus recognizing T-cells after the treatment with the oncolytic adenovirus Ad5/3-hTERT-E1A-hCD40L. Total PBMCs were isolated and pulsed with an adenovirus 5 penton-derived peptide pool to assess the activation of adenovirus-specific cytotoxic T-lymphocytes with interferon gamma ELISPOT.

FIG. 10 shows the results of the analysis of patient samples for either Th1 induced cytokines: interferon-γ (IFN-γ), tumor necrosis factor-α (TNF-α) and interleukin-2 (IL-2) or Th2 cytokines: interleukin-4 (IL-4), interleukin-5 (IL-5) and interleukin 10 (IL-10) with Becton-Dickinson cytokine multiplex bead array system (BD FACSArray; BD Biosciences, San Jose, Calif.) according to the manufacturer's instructions. On the left side of FIG. 10 are presented Th1 induced cytokines and on the right side the Th2 induced cytokines. Before=serum sample taken before the virus was given; 1 month=serum sample taken 1 month after the virus treatment; 2 months=serum samples taken 2 months after the virus treatment.

FIG. 11 shows the results of the analysis of patient serum samples for Th1 cytokines: interferon-γ (IFN-γ), tumor necrosis factor-α (TNF-α) and interleukin-2 (IL-2) and for Th2 cytokines: interleukin-4 (IL-4), interleukin-5 (IL-5) and interleukin 10 (IL-10) with a Becton-Dickinson cytokine multiplex bead array system (BD FACSArray; BD Biosciences, San Jose, Calif.) according to the manufacturer's instructions. Cytokines levels are reported relative to their baseline level, which was set as 1 and a ratio between Th1/Th2 was calculated for each time point. 1 month=serum sample taken 1 month after the virus treatment; 2 months=serum samples taken 2 months after the virus treatment.

FIG. 12 shows the results of the assessment of the activation of adenovirus-specific (12A) and tumor-specific (12B) cytotoxic T-lymphocytes with interferon gamma ELISPOT without pre-stimulation. Stars indicate the days of virus administration. PBMCs were collected immediately prior to virus injection.

FIG. 13 shows serum levels for IL-6 (13A), IL-8 (13B), IL-10 (13C), IL-12 (13D), TNF-alpha (13E), and INF-gamma (13F) assessed before and after the treatment. Data are presented as median±SD.

FIG. 14 shows the effects on anti-tumor and anti-adenoviral immunity. Pre-stimulated and clonally expanded PBMCs were pulsed either with tumor-derived peptide pool (specified for each patient according to tumor type) or adenovirus-derived peptide pool. The relative numbers of TNF-alpha /INF-gamma double positive tumor-specific CD8+ T-cells (14A), tumor-specific CD4+ T-cells (14B), and adenovirus-specific CD4+ T-cells (14C) were assessed with intracellular cytokine staining. Stars indicate the days of virus administration. PBMCs were collected immediately prior to virus injection.

FIG. 15 shows the local levels of soluble CD40L (sCD40L; 15A) and RANTES (15B) in the malignant ascites fluid compared to systemic levels of these cytokines. High amounts of virus particles (VP) were found in the malignant ascites fluid (15C) and in cells isolated from ascites (15D) on day 28 after virus treatment, whereas no virus was detected in the serum on the same day.

FIG. 16 shows the assessment of sCD40L and RANTES concentrations in the sera of 9 cancer patients before (baseline) and at several time points after the treatment. Data are presented as median±SD.

FIG. 17 shows the overall survival (A, p=0.007) and progression free survival (B, p=0.146) of oncolytic adenovirus Ad5/3-hTERT-E1A-hCD40L (referred to as CGTG-401 in FIG. 17) treated patients by Kaplan-Meier analysis. Historical controls had similar inclusion and exclusion criteria and were treated with an oncolytic adenovirus with an identical capsid but lacking a transgene (Ad5/3-cox2L-D24).

### DETAILED DESCRIPTION OF THE INVENTION

### Adenoviral Vector

In Ad5, as well as in other adenoviruses, an icosahedral capsid consists of three major proteins: hexon (II), penton base (III), and a knobbed fiber (IV), along with minor proteins: VI, VIII, IX, IIIa, and IVa2 (Russell W. C., J General Virol 2000; 81:2573-2604). Proteins VII, small peptide mu, and a terminal protein (TP) are associated with DNA. Protein V provides a structural link to the capsid via protein VI. Virus encoded protease is needed for processing some structural proteins.

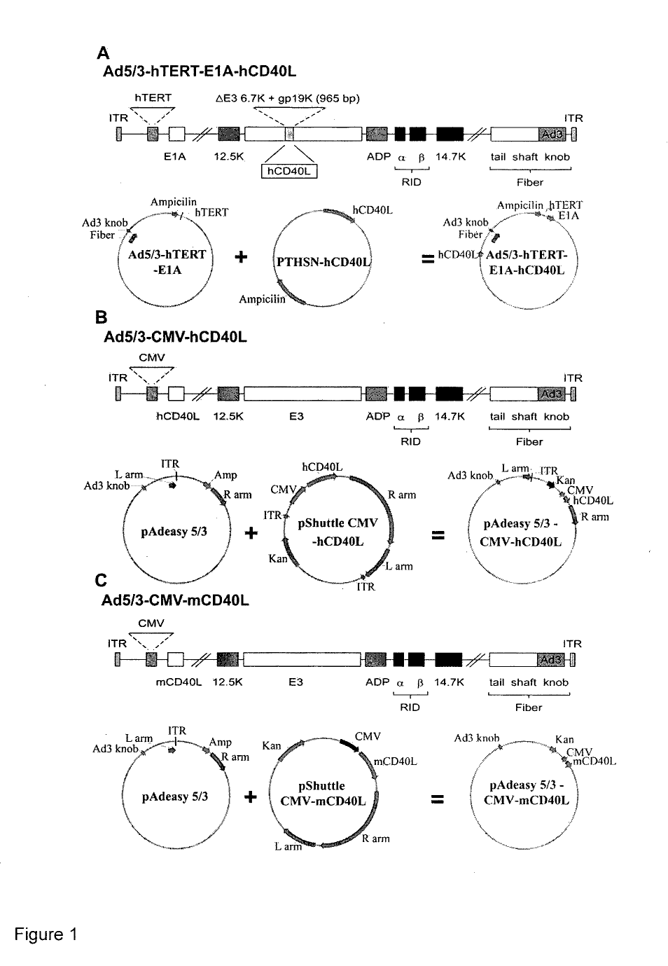

The oncolytic adenoviral vector of the present invention is based on an adenovirus serotype 5 (Ad5) nucleic acid backbone comprising a capsid modification, such as an adenovirus serotype 3 (Ad3) knob (Ad5/3 capsid chimerism), a nucleic acid sequence encoding a tumor specific human telomerase reverse transcriptase (hTERT) promoter (SEQ ID. NO:1) upstream of the E1 region; and a nucleic acid sequence encoding human CD40L (SEQ ID. NO:2) in place of the deleted gp19k/6.7K sequences (965 base pairs) in the E3 region (FIG. 1a). In a preferred embodiment of the invention, the adenoviral vector is based on a human adenovirus. The CD40L sequence used here is distinct from the human genomic sequence (NG_{—}007280.1) to facilitate detection from human samples. Therefore, the present invention discloses a unique sequence variant OF CD40L.

The Ad5 genome contains early (E1-4), intermediate (IX and IVa2) and late (L1-5) genes flanked by left and right inverted terminal repeats (LITR and RITR, respectively), which contain the sequences required for the DNA replication. The genome also contains packaging signal (ψ) and major late promoter (MLP).

Transcription of the early gene E1A starts the replication cycle followed by expression of E1B, E2A, E2B, E3 and E4. E1 proteins modulate cellular metabolism in a way that makes a cell more susceptible to virus replication. For example they interfere with NF-κB, p53, and pRb-proteins. E1A and E1B function together in inhibiting apoptosis. E2 (E2A and E2B) and E4 gene products mediate DNA replication and E4 products also effect the virus RNA metabolism and prevent the host protein synthesis. The E3 gene products are responsible for defending against the host immune system, enhancing cell lysis, and releasing of virus progeny (Russell W. C., J General Virol 2000; 81:2573-2604).

Intermediate genes IX and IVa2 encode minor proteins of the viral capsid. Expression of the late genes L1-5, which lead to production of the virus structural components, encapsidation and maturation of the virus particles in the nucleus, is influenced by MLP (Russell W. C., J General Virol 2000; 81:2573-2604).

Compared to a wild type adenovirus genome, the adenoviral vector of the invention comprises hTERT promoter in the E1 region, specifically upstream of the E1A region, lacks gp19k and 6.7K in the E3 region, and comprises a capsid modification in the fiber of the virus. In a preferred embodiment of the invention, in addition to amended/partial regions E1 and E3, the oncolytic adenoviral vector of the invention further comprises one or more regions selected from a group consisting of E2, E4, and late regions. In a preferred embodiment of the invention, the oncolytic adenoviral vector comprises the following regions: a left ITR, partial E1, pIX, plVa2, E2, VA1, VA2, L1, L2, L3, L4, partial E3, L5, E4, and a right ITR. The regions may be in any order in the vector, but in a preferred embodiment of the invention, the regions are in a sequential order in the 5′ to 3′ direction. Open reading frames (ORFs) may be in the same DNA strand or in different DNA strands. In a preferred embodiment of the invention, the E1 region comprises a viral packaging signal.

As used herein, expression “adenovirus serotype 5 (Ad5) nucleic acid backbone” refers to the genome or partial genome of Ad5, which comprises one or several regions selected from a group consisting of partial E1, pIX, plVa2, E2, VA1, VA2, L1, L2, L3, L4, partial E3, L5 and E4 of Ad5 origin. In a preferred embodiment, the vector of the invention comprises a nucleic acid backbone of Ad5 with a portion of Ad3 (e.g., a part of the capsid structure).

As used herein, expression “partial” region refers to a region, which lacks any part compared to a corresponding wild type region. For instance “partial E3” refers to E3 region lacking gp19k/6.7K.

As used herein, expressions “VA1” and “VA2” refer to virus associated RNAs 1 and 2, which are transcribed by the adenovirus but are not translated. VA1 and VA2 have a role in combating cellular defense mechanisms.

As used herein, expression “a viral packaging signal” refers to a part of virus DNA, which consists of a series of AT-rich sequences and governs the encapsidation process.

The E3 region is nonessential for viral replication in vitro, but the E3 proteins have an important role in the regulation of host immune response, i.e. in the inhibition of both innate and specific immune responses. The gp19k/6.7K deletion in E3 refers to a deletion of 965 base pairs from the adenoviral E3A region. In a resulting adenoviral construct, both gp19k and 6.7K genes are deleted (Kanerva A et al., Gene Therapy 2005; 12: 87-94). The gp19k gene product is known to bind and sequester major histocompatibility complex I (MHC1) molecules in the endoplasmic reticulum, and to prevent the recognition of infected cells by cytotoxic T-lymphocytes. Since many tumors are deficient in MHC1, deletion of gp19k increases tumor selectivity of viruses (virus is cleared faster than wild type virus from normal cells but there is no difference in tumor cells). 6.7K proteins are expressed on cellular surfaces and they take part in downregulating TNF-related apoptosis inducing ligand (TRAIL) receptor 2.

In the present invention, the CD40L transgene is placed into a gp19k/6.7 k deleted E3 region, under the E3 promoter. This restricts transgene expression to tumor cells that allow replication of the virus and subsequent activation of the E3 promoter. The E3 promoter may be any exogenous or endogenous promoter known in the art, preferably endogenous promoter. In a preferred embodiment of the invention, a nucleic acid sequence encoding CD40L is under the control of the viral E3 promoter.

The gp19k deletion is particularly useful in the context of CD40L expression as it can enhance MHC1 presentation of tumor epitopes in such tumors that retain this capacity. In this context, stimulation of APC and T-cells by CD40L can yield the optimum benefit.

CD40L potentiates the immune response by acting through various mechanisms including recruitment of cytotoxic T-cell, natural killer (NK) cells, stimulation of antigen presenting cells (APC) and down-regulation of suppressive cells such as regulatory T-cells. APC can then recruit, activate and target T-cells towards the tumor. The nucleotide sequence encoding CD40L may be from any animal, such as a human, ape, rat, mouse, hamster, dog or cat depending on the subject to be treated, but preferably CD40L is encoded by a human sequence in the context of treatment of humans. The nucleotide sequence encoding CD40L may be modified in order to improve the effects of CD40L, or unmodified, i.e. of a wild type. In a preferred embodiment of the invention, a nucleic acid sequence encoding CD40L is modified with one nucleotide from the wild type sequence to allow specific detection from human samples.

Insertion of exogenous elements may enhance effects of vectors in target cells. The use of exogenous tissue or tumor-specific promoters is common in recombinant adenoviral vectors. The viral replication is restricted to target cells by the use of hTERT or variants of hTERT to control the E1A region. In a preferred embodiment of the invention hTERT is placed upstream of E1A, but in addition to or alternatively, other genes such as E1B or E4 can also be regulated. “Upstream” refers to immediately before the E1 region in the direction of expression. Exogenous insulators i.e. blocking elements against unspecific enhancers, the left ITR, the native E1A promoter or chromatin proteins may also be included in recombinant adenoviral vectors. Any additional components or modifications may optionally be used but are not obligatory in the vectors of the present invention.

The oncolytic adenoviral vector of the invention comprises a capsid modification. Most adults have been exposed to the most widely used adenovirus serotype Ad5 and therefore, the immune system can rapidly produce neutralizing antibodies (NAb) against them. In fact, the prevalence of anti-Ad5 NAb may be up to 50%. It has been shown that NAb can be induced against most of the multiple immunogenic proteins of the adenoviral capsid, and on the other hand, it has been shown that even small changes in the Ad5 fiber knob can allow escape from capsid-specific NAb. Modification of the knob is therefore important for retaining or increasing gene delivery in the contact of adenoviral use in humans.

Furthermore, Ad5 is known to bind to the receptor called CAR via the knob portion of the fiber, and modifications of this knob portion or fiber may improve the entry to the target cell and cause enhanced oncolysis in many or most cancers (Ranki T. et al., Int J Cancer 2007; 121:165-174). Indeed, capsid-modified adenoviruses are advantageous tools for improved gene delivery to cancer cells.

As used herein “capsid” refers to the protein shell of the virus, which includes hexon, fiber and penton base proteins. Any capsid modification i.e. modification of hexon, fiber and/or penton base proteins known in the art, which improves delivery of the virus to the tumor cell, may be utilized in the present invention. Modifications may be genetic and/or physical modifications and include but are not limited to modifications for incorporating ligands, which recognize specific cellular receptors and/or block native receptor binding, for replacing the fiber or knob domain of an adenoviral vector with a knob of other adenovirus (chimerism) and for adding specific molecules (e.g., fibroblast growth factor 2, FGF2) to adenoviruses. Therefore, capsid modifications include but are not limited to incorporation of small peptide motif(s), peptide(s), chimerism(s) or mutation(s) into the fiber (e.g., into the knob, tail or shaft part), hexon and/or penton base. In a preferred embodiment of the invention, the capsid modification is Ad5/3 chimerism, insertion of an integrin binding (RGD) region and/or heparin sulphate binding polylysine modification into the fiber. In a specific embodiment of the invention, the capsid modification is Ad5/3 chimerism.

As used herein, “Ad5/3 chimerism” of the capsid refers to a chimerism, wherein the knob part of the fiber is from Ad serotype 3, and the rest of the fiber is from Ad serotype 5.

The vector of the invention may also comprise other modifications, such as modifications of the E1B region.

As used herein, “RGD” refers to the arginine-glycine-aspartic acid (RGD) motif, which is exposed on the penton base and interacts with cellular α-v-β-integrins supporting adenovirus internalization. In a preferred embodiment of the invention, the capsid modification is a RGD-4C modification. “RGD-4C modification” refers to an insertion of a heterologous integrin binding RGD-4C motif in the HI loop of the fiber knob domain. 4C refers to the four cysteins, which form sulphur bridges in RGD-4C. Construction of recombinant Ad5 fiber gene encoding the fiber with the RGD-4C peptide is described in detail for example in the article of Dmitriev I. et al. (Journal of Virology 1998; 72:9706-9713).

As used herein, “heparan sulphate binding polylysine modification” refers to addition of a stretch of seven lysines to the fiber knob c-terminus.

Expression cassettes are used for expressing transgenes in a target, such as a cell, by utilizing vectors. As used herein, the expression “expression cassette” refers to a DNA vector or a part thereof comprising nucleotide sequences, which encode cDNAs or genes, and nucleotide sequences, which control and/or regulate the expression of said cDNAs or genes. Similar or different expression cassettes may be inserted to one vector or to several different vectors. Ad5 vectors of the present invention may comprise either several or one expression cassettes. However, only one expression cassette is adequate. In a preferred embodiment of the invention, the oncolytic adenoviral vector comprises at least one expression cassette. In a preferred embodiment of the invention, the oncolytic adenoviral vector comprises only one expression cassette.

A cell comprising the adenoviral vector of the invention may be any cell such as a eukaryotic cell, bacterial cell, animal cell, human cell, mouse cell etc. A cell may be an in vitro, ex vivo or in vivo cell. For example, the cell may be used for producing the adenoviral vector in vitro, ex vivo or in vivo, or the cell may be a target, such as a tumor cell, which has been infected with the adenoviral vector.

In a method of producing CD40L in a cell, a vehicle comprising the vector of the invention is carried into a cell and the CD40L gene is expressed and the protein is translated and secreted in a paracrine manner. “A vehicle” may be any viral vector, plasmid or other tool, such as a particle, which is able to deliver the vector of the invention to a target cell. Any conventional method known in the art can be used for delivering the vector to the cell.

Tumor specific immune response may be increased in a subject by the present invention. Cytotoxic T cells and/or natural killer cells are stimulated and recruited to the tumor area as a consequence of CD40L expression. In a preferred embodiment of the invention, the amount of natural killer and/or cytotoxic T cells is increased in a target cell or tissue. In order to follow or study the effects of the invention, various markers of immune response (e.g. inflammatory markers) may be determined. The most common markers include but are not limited to increase in pro-inflammatory cytokines, tumor or adenovirus specific cytotoxic T-cells, recruitment and activation of antigen presenting cells or increase in size of local lymph nodes. The levels of these markers may be studied according to any conventional methods known in the art, including but not limited to those utilizing antibodies, probes, primers etc., such as ELISPOT assay, tetramer analysis, pentamer analysis and analysis of different cell types in blood or in tumors.

### Cancer

The oncolytic adenoviral vectors of the invention have been constructed for replication competence in cells, which express human telomerase reverse transcriptase (hTERT), which is the catalytic subdomain of human telomerase. These include over 85% of human tumors, which are found to upregulate expression of the hTERT gene and its promoter, whereas most normal adult somatic cells are devoid of telomerase or transiently express very low levels of the enzyme (Shay and Bacchetti 1997, Eur J Cancer 33:787-791).

Any cancers or tumors, including both malignant and benign tumors as well as primary tumors and metastasis may be targets of gene therapies, as long as they express hTERT. In a specific embodiment of the invention the cancer is any solid tumor. In a preferred embodiment of the invention, the cancer is selected from a group consisting of nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, oligodendroglioma, neuroblastoma, meningioma, spinal cord tumor, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, esophagus cancer, gall bladder cancer, head cancer, eye cancer, neck cancer, kidney cancer, Wilms' tumor, Kaposi's sarcoma, prostate cancer, testicular cancer, Hodgkin's disease, non-Hodgkin's lymphoma, skin cancer, mesothelioma, multiple myeloma, ovarian cancer, endocrine pancreatic cancer, glucagonoma, pancreatic cancer, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer, and tonsil cancer.

### Pharmaceutical Composition

A pharmaceutical composition of the invention comprises at least one type of the vectors of the invention. Furthermore, the composition may comprise at least two, three or four different vectors of the invention. In addition to the vector of the invention, a pharmaceutical composition may also comprise any other vectors, such as other adenoviral vectors, such as those described in US2010166799 A1, other therapeutically effective agents, any other agents, such as pharmaceutically acceptable carriers, buffers, excipients, adjuvants, antiseptics, filling, stabilizing or thickening agents, and/or any components normally found in corresponding products.

The pharmaceutical composition may be in any form, such as in a solid, semisolid or liquid form, suitable for administration. A formulation can be selected from a group consisting of, but not limited to, solutions, emulsions, suspensions, tablets, pellets and capsules.

In a preferred embodiment of the invention, the oncolytic adenoviral vector or pharmaceutical composition acts as an in situ cancer vaccine. As used herein “in situ cancer vaccine” refers to a cancer vaccine, which both kills tumor cells and also increases the immune response against tumor cells. Virus replication is a strong danger signal to the immune system (=needed for a TH1 type response), and thus acts as a powerful co-stimulatory factor for CD40L mediated maturation and activation of APCs, and recruitment of NK cells. Suppression of regulatory cells also helps in this regard. Tumor cell lysis also helps to present tumor fragments and epitopes to APCs and further co-stimulation is produced by inflammation. Thus, an epitope independent (i.e., not HLA restricted) response is produced in the context of each tumor and therefore takes place in situ. Tumor specific immune response is activated in the target cells allowing thereafter antitumor activities to occur on the whole subject level, e.g., in distant metastases. The effective dose of vectors depends on many factors including the subject in need of the treatment, the tumor type, the location of the tumor and the stage of the tumor. The dose may vary for example from about 10⁸ viral particles (VP) to about 10¹⁴ VP, preferably from about 5×10⁹ VP to about 10¹³ VP and more preferably from about 8×10⁹ VP to about 10¹² VP. In one specific embodiment of the invention the dose is in the range of about 5×10¹⁰-5×10¹¹ VP.

The pharmaceutical compositions may be produced by any conventional processes known in the art, for example by utilizing any one of the following: batch, fed-batch and perfusion culture modes, column-chromatography purification, CsCI gradient purification and perfusion modes with low-shear cell retention devices.

### Administration

The vector or pharmaceutical composition of the invention may be administered to any eukaryotic subject selected from a group consisting of plants, animals and human beings. In a preferred embodiment of the invention, the subject is a human or an animal. An animal may be selected from a group consisting of pets, domestic animals and production animals.

Any conventional method may be used for administration of the vector or composition to a subject. The route of administration depends on the formulation or form of the composition, the disease, the location of tumors, the patient, co-morbidities and other factors. In a preferred embodiment of the invention, the administration is conducted through an intratumoral, intramuscular, intra-arterial, intravenous, intrapleural, intravesicular, intracavitary or peritoneal injection, or an oral administration.

Only one administration of oncolytic adenoviral vectors of the invention may have therapeutic effects. However, in a preferred embodiment of the invention, oncolytic adenoviral vectors or pharmaceutical compositions are administered several times during the treatment period. Oncolytic adenoviral vectors or pharmaceutical compositions may be administered for example from 1 to 10 times in the first 2 weeks, 4 weeks, monthly or during the treatment period. In one embodiment of the invention, administration is done three to seven times in the first 2 weeks, then at 4 weeks and then monthly. In a specific embodiment of the invention, administration is done four times in the first 2 weeks, then at 4 weeks and then monthly. The length of the treatment period may vary, and for example may last from two to 12 months or more.

Additionally, the administration of the oncolytic adenoviral vectors of the invention can preferably be combined to the administration of other oncolytic adenoviral vectors, such as those described in US2010166799 A1. The administration can be simultaneous or sequential.

In order to avoid neutralizing antibodies in a subject, the vectors of the invention may vary between treatments. In a preferred embodiment of the invention, the oncolytic adenoviral vector having a different fiber knob of the capsid compared to the vector of the earlier treatment is administered to a subject. As used herein “fiber knob of the capsid” refers to the knob part of the fiber protein (FIG. 1a). Alternatively, the entire capsid of the virus may be switched to that of a different serotype.

The gene therapy of the invention is effective alone, but combination of adenoviral gene therapy with any other therapies, such as traditional therapy, may be more effective than either one alone. For example, each agent of the combination therapy may work independently in the tumor tissue, the adenoviral vectors may sensitize cells to chemotherapy or radiotherapy and/or chemotherapeutic agents may enhance the level of virus replication or affect the receptor status of the target cells. Alternatively, the combination may modulate the immune system of the subject in a way that is beneficial for the efficacy of the treatment. For example, chemotherapy could be used to downregulate suppressive cells such as regulatory T-cells. The agents of combination therapy may be administered simultaneously or sequentially. In a preferred embodiment of this invention, patients receive simultaneous cyclophosphamide to enhance the immunological effect of the treatment.

In a preferred embodiment of the invention, the method or use further comprises administration of concurrent radiotherapy to a subject. In another preferred embodiment of the invention, the method or use further comprises administration of concurrent chemotherapy to a subject. In yet another preferred embodiment of the invention, the method or use further comprises administration of other oncolytic adenovirus or vacciniavirus vectors to a subject. The administration of vectors can be simultaneous or sequential.

As used herein “concurrent” refers to a therapy, which has been administered before, after or simultaneously with the gene therapy of the invention. The period for a concurrent therapy may vary from minutes to several weeks. Preferably the concurrent therapy lasts for some hours. In one embodiment, cyclochosphamide is administered both as an intravenous bonus and orally in a metronomic fashion.

Agents suitable for combination therapy include but are not limited to All-trans retinoic acid, Azacitidine, Azathioprine, Bleomycin, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Mechlorethamine, Mercaptopurine, Methotrexate, Mitoxantrone, Oxaliplatin, Paclitaxel, Pemetrexed, Temozolomide, Teniposide, Tioguanine, Valrubicin, Vinblastine, Vincristine, Vindesine and Vinorelbine.

In a preferred embodiment of the invention, the method or use further comprises administration of verapamil or another calcium channel blocker to a subject. “Calcium channel blocker” refers to a class of drugs and natural substances which disrupt the conduction of calcium channels, and it may be selected from a group consisting of verapamil, dihydropyridines, gallopamil, diltiazem, mibefradil, bepridil, fluspirilene and fendiline.

In a preferred embodiment of the invention, the method or use further comprises administration of autophagy inducing agents to a subject. Autophagy refers to a catabolic process involving the degradation of a cell's own components through the lysosomal machinery. “Autophagy inducing agents” refer to agents capable of inducing autophagy and may be selected from a group consisting of, but not limited to, mTOR inhibitors, PI3K inhibitors, lithium, tamoxifen, chloroquine, bafilomycin, temsirolimus, sirolimus and temozolomide. In a specific embodiment of the invention, the method further comprises administration of temozolomide to a subject. Temozolomide may be either oral or intravenous temozolomide. Autophagy inducing agents may be combined with immunomodulatory agents. In one embodiment, oncolytic adenovirus coding for CD40L is combined with both temozolomide and cyclophosphamide.

In one embodiment of the invention, the method or use further comprises administration of chemotherapy or anti-CD20 therapy or other approaches for blocking of neutralizing antibodies. “Anti-CD20 therapy” refers to agents capable of killing CD20 positive cells, and may be selected from a group consisting of rituximab and other anti-CD20 monoclonal antibodies. “Approaches for blocking of neutralizing antibodies” refers to agents capable of inhibiting the generation of anti-viral antibodies that normally result from infection and may be selected from a group consisting of different chemotherapeutics, immunomodulatory substances, corticoids and other drugs. These substances may be selected from a group consisting of, but not limited to, cyclophosphamide, cyclosporin, azathioprine, methylprenisolone, etoposide, CD40L, CTLA41g4, FK506 (tacrolismus), IL-12, IFN-gamma, interleukin 10, anti-CD8, anti-CD4 antibodies, myeloablation and oral adenoviral proteins.

The approach described in this application can also be combined with molecules capable of overcoming neutralizing antibodies. Such agents include liposomes, lipoplexes and polyethylene glycol, which can be mixed with the virus. Alternatively, neutralizing antibodies can be removed with an immunopheresis column consisting of adenoviral capsid proteins.

The oncolytic adenoviral vector of the invention induces virion mediated oncolysis of tumor cells and activates human immune response against tumor cells. In a preferred embodiment of the invention, the method or use further comprises administration of substances capable to down-regulating regulatory T-cells in a subject. “Substances capable to down-regulating regulatory T-cells” refers to agents that reduce the amount of cells identified as T-suppressor or Regulatory T-cells. These cells have been identified as featuring one or many of the following immunophenotypic markers: CD4+, CD25+, FoxP3+, CD127− and GITR+. Such agents reducing T-suppressor or Regulatory T-cells may be selected from a group consisting of anti-CD25 antibodies or chemotherapeutics.

In a preferred embodiment of the invention, the method or use further comprises administration of cyclophosphamide to a subject. Cyclophosphamide is a common chemotherapeutic agent, which has also been used in some autoimmune disorders. In the present invention, cyclophosphamide can be used to enhance viral replication and the effects of CD40L induced stimulation of NK and cytotoxic T-cells for enhanced immune response against the tumor. It can be used as intravenous bolus doses or low-dose oral metronomic administration or their combination.

Any method or use of the invention may be either in vivo, ex vivo or in vitro method or use.

One perceived limitation of the use of adenoviruses for the treatment of cancer is their immunogenicity. However, as the immune system of cancer patients has failed to eliminate the tumor because of the immunosuppressive nature of the tumor environment, the immunogenicity of adenoviruses becomes an advantage. In the present invention, this effect has been potentiated by retaining replication competence and arming with an immunostimulatory molecule, CD40L. The adenovirus vectors of the present invention feature four important aspects. Tumor transduction is improved by capsid modification, such as serotype chimerism with the Ad3 knob in an otherwise Ad5 capsid. Tumor selectivity is achieved by inserting the hTERT promoter in front of E1A. Recruitment and stimulation of antigen presenting cells for induction of a Th1-type cytotoxic T-cell response is achieved by arming the virus with CD40L. Finally, CD40L can also cause apoptosis of CD40+ tumors.

Adenoviruses of the present invention were found effective in inducing CD40L expression both in CD40+ and CD40− cells. In an oncolytic platform, which ensures that transduced tumor cells are ultimately killed by oncolysis, the secretion or release of CD40L from lysing cells will cause an apoptotic bystander effect on tumor cells near-by. However, it is believed that the main advantage of the use of CD40L is the immunostimulatory effect.

It has been shown that an oncolytic adenovirus Ad5/3-hTERT-E1A has significantly higher oncolytic potency compared to that of wild type Ad5 (Bauerschmitz G J, et al., Cancer Res 2008; 68:5533-9). In the same study, when hTERT selectivity was compared in a panel of oncolytic adenoviruses featuring different tissue specific promoters, such as α-lactalbumin, cyclo-oxygenase or multidrug resistance protein, Ad5/3-hTERT-E1A displayed best results in vitro and significant antitumor effect in vivo. Thus, Ad5/3-hTERT-E1A is an ambitious control virus for the adenoviruses of the present invention.

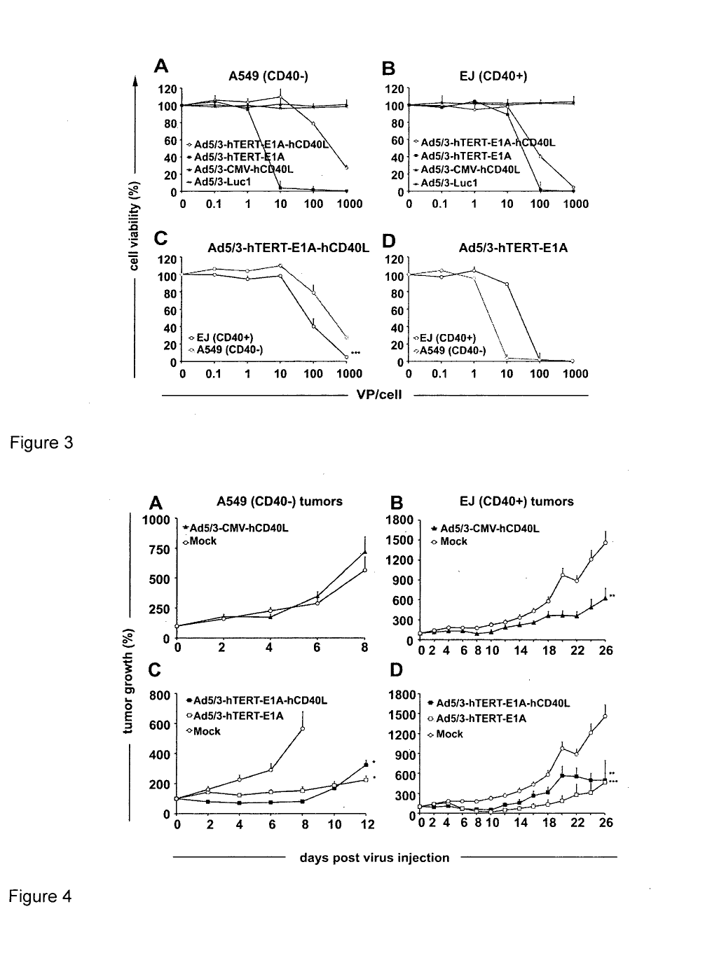

When the oncolytic adenoviruses of the present invention, as exemplified by Ad5/3-hTERT-E1A-hCD40L, were compared to the non-armed oncolytic adenovirus Ad5/3-hTERT-E1A, it was found that both viruses were equally effective with regard to oncolytic potency in vivo (FIGS. 4c, 4d). This was an important finding as expression of transgenes can sometimes inhibit the potency of viruses and Ad5/3-hTERT-E1A-hCD40L was slower than Ad5/3-hTERT-E1A on A549 cells in vitro. In vitro Ad5/3-hTERT-E1A-hCD40L had more antitumor activity on CD40+EJ cells than on CD40− A549 cells, while the opposite was true for Ad5/3-hTERT-E1A (FIGS. 3c, 3d).

Although the biggest utility of oncolytic adenoviruses of the invention, such as Ad5/3-hTERT-E1A-hCD40L, might be in the context of CD40+ tumors, where all three anti-tumor activities (oncolysis, apoptosis, immune stimulation) would contribute, it is believed that the potential utility of the adenoviruses of the invention is not restricted to CD40+ tumors. There are reports demonstrating that CD40L activates antigen presenting cells even when the tumor is CD40− (Noguchi M, et al., Cancer Gene Ther 2001; 8:421-9; Sun Y, et al., Gene Ther 2000; 7:1467-76. As even unarmed oncolytic adenoviruses have shown utility in humans, the oncolytic adenoviruses carrying hCD40L might represent an improvement regardless of CD40 status of the tumor.

Clinical and preclinical work in the field of tumor immunology and vaccine development over the past two decades has demonstrated that induction of an antitumor immune response can be achieved with several approaches. Unfortunately, however, approaches aiming at induction of immune responses have not been very effective for patients with advanced and highly immunosuppressive tumors. Instead, the first successful immunotherapeutics feature either trained and stimulated T-cells (to overcome tumor mediated immune suppression) or antibodies capable to down-regulating immune suppressivity (Motohashi et al 2006 Clin Cancer Res 12:6079-86; Hodi et al 2008 Nature Clinical Practice Oncology 5:557-561). Many investigators also use preconditioning to “make room” for activated T-cells and reduce immune suppressive cells. Thus, a critical lesson is that breaking the immunological tolerance acquired by tumors may be required for successful immunotherapy.

However, the adenovirus vectors of the present invention showed antitumor responses in patients with refractory and immune suppressive disease and these effects were related to induction of immunity and Th2 to Th1 switch.

In conclusion, with the oncolytic CD40L expressing adenovirus vectors of the invention, including Ad5/3-hTERT-E1A-hCD40L, a significant anti-tumor effect is obtained. An important part of the effect is induction of a Th1 type immune response which results in accumulation of cytotoxic T cells at the tumor site and their activation.

The present invention is illustrated by the following examples, which are not intended to be limiting in any way.

### EXAMPLES

### Animals

All animal protocols were reviewed and approved by the Experimental Animal Committee of the University of Helsinki and the Provincial Government of Southern Finland. C57Black mice and NMRI nude mice were obtained from Taconic (Ejby, Denmark) at 4 to 5 weeks of age and quarantined at least for 1 week prior to the study. Health status of the mice was frequently monitored and soon as any sign of pain or distress was evident they were killed.

### Cell Lines

For the immune deficient models 10⁶ A549 (human lung adenocarcinoma cell line available from American Type Culture Collection, ATCC, 10801 University Boulevard, Manassas, Va. 20110-2209, USA) or EJ cells (human bladder carcinoma cell line, kindly provided by Dr. Angelina Loskog, University of Uppsala, Sweden) were used. Ramos-Blue™ cell line was from InvivoGen (San Diego, Calif., USA).

For the immunocompetent model 5×10⁵ MB49 cells (mouse bladder carcinoma cell line, kindly provided by Dr. Angelina Loskog, University of Uppsala, Sweden) were injected subcutaneously on shaved flanks of C57Black mice (n=7mice/group). Virus was injected three times intratumorally at the dose of 3×10⁸VP/tumor on days 0, 2 and 4, when tumors reached the size of approximately 5×5 mm.

### Statistical Analysis

Two tailed Student's t-test was used and a p-value of <0.05 was considered as significant. Survival data was processed with Kaplan-Meier analysis.

### Example 1

### Cloning of Ad5/3-hTERT-E1A-hCD40L, Ad5/3-CMV-hCD40L and Ad5/3-CMV-mCD40L

Ad5/3-hTERT-E1A-hCD40L (SEQ ID. NO:5) was generated and amplified using standard adenovirus preparation techniques (Kanerva A, et al., Mol Ther 2002; 5:695-704; Bauerschmitz G J, et al., Mol Ther 2006; 14:164-74; Kanerva A and Hemminki A., Int J Cancer 2004; 110:475-80; Volk A L, et al., Cancer Biol Ther 2003; 2:511-5). Briefly, human CD40L cDNA (kind gift from Prof Eliopoulos, University of Crete, Heraklion, Greece) was amplified by a polymerase chain reaction (PCR) with specific primers (forward primer: TTTAACATCTCTCCCTCTGTGATT; SEQ ID NO:3 and reverse primer: TATAAATGGAGCTTGACTCGAAG; SEQ ID NO:4) featuring insertion of specific restriction sites SunI/MunI. The PCR amplification product was then subcloned into pTHSN (Kanerva A., et al., Gene Ther 2005; 12:87-94) and subsequently recombined with an pAd5/3-hTERT-E1A (Bauerschmitz G J, et al., Cancer Res 2008; 68:5533-9) to generate pAd5/3-hTERT-E1A-hCD40L. This plasmid was linearized with PacI and transfected into A549 cells for amplification and rescue.

All phases of the cloning were confirmed with PCR and multiple restriction digestions. The shuttle plasmid pTHSN-hCD40L was sequenced. The absence of wild type E1 was confirmed with PCR. The E1 region, transgene and fiber were checked in the final virus with sequencing and PCR. All phases of the virus production, including transfection, were done on A549 cells to avoid risk of wild type recombination, as described before (Kanerva A et al. 2003, Mol Ther 8, 449-58; Bauerschmitz G J et al. 2006, Mol Ther 14, 164-74). hCD40L is under the E3 promoter (specifically under endogenous viral E3A gene expression control elements), which results in replication associated transgene expression, which starts about 8 h after infection. E3 is intact except for deletion of 6.7K/gp19K. FIG. 1a shows the structure of pAd5/3-hTERT-E1A-hCD40L.

For construction of non-replicating adenoviruses Ad5/3-CMV-hCD40L and Ad5/3-CMV-mCD40L, expression cassettes with either hCD40L or mCD40L were inserted into the multiple cloning site of pShuttle-CMV plasmid (Stratagene, La Jolla, Calif., USA). The shuttle plasmids were recombined with pAdeasy-1.5/3 plasmid (Krasnykh V N, et al., J Virol 1996; 70:6839-46), which carries the whole adenovirus genome, and the resulting rescue plasmids were transfected to 293 cells (human transformed embryonic kidney cell line available from Microbix, Toronto, Ontario; Canada) to generate Ad5/3-CMV-hCD40L and Ad5/3-CMV-mCD40L. FIGS. 1b and 1c show the structures and cloning of Ad5/3-CMV-hCD40L and Ad5/3-CMV-mCD40L, respectively.

Control vectors Ad5/3-Luc1 (Kanerva A, et al., Clin Cancer Res 2002; 8:275-80) and Ad5/3-hTERT-E1A (Bauerschmitz G J, et al., Cancer Res 2008; 68:5533-9) have been previously reported. The VP to plaque forming units ratios for Ad5/3-Luc1, Ad5/3-hTERT-E1A, Ad5/3-hTERT-E1A-hCD40L, Ad5/3-CMV-hCD40L, and Ad5/3-CMV-mCD40L, were 25, 31, 200, 138, and 86, respectively.

### Example 2

### Expression and Functionality of the Constructed Adenoviruses: In Vitro and In Vivo

Flow-cytometry and enzyme-linked immunosorbent assay (ELISA) were used to study the hCD40L-expression. For a flow-cytometry analysis, human embryonic kidney 293 cells were infected with Ad5/3-hTERT-E1A-hCD40L or Ad5/3-CMV-hCD40L using 10 VP/cell in growth media containing 2% fetal calf serum (FCS). Control cells (mock) were treated with 2% Dulbecco's modified Eagle's medium (DMEM) alone. Twenty four hours later the cells were stained either with an hCD40L-FITC (555699, BD Biosciences Pharmingen Franklin Lakes, N.J.) antibody for 30 minutes or with isotype control (IC) for measuring background fluorescence from cellular autofluorescence and nonantigen-specific binding. Flow cytometry analysis was performed on BDLSR (BD Biosciences, Franklin Lakes, N.J.).

For an ELISA analysis, A549 xenografts and syngeneic MB49 tumors were induced and treated either with Ad5/3-hTERT-E1A-hCD40L, Ad5/3-CMV-hCD40L, or Ad5/3-CMV-mCD40L as above. Blood samples were taken on days 4, 8 and 12 after the first virus injection. For analyzing hCD40L in the serum from mice treated with Ad5/3-CMV-hCD40L, blood was collected only twice (day 4 and 8) due to rapid tumor growth and animals had to be killed on day 8. The hCD40L and mCD40L concentrations in the serum were determined with Human CD40 Ligand ELISA kit (ELH-CD40L-001, RayBiotech Inc, Norcross Ga., USA) and Mouse sCD40L ELISA kit (BMS6010, Bender Medsytems, Austria) according to the manufacturer's protocol.

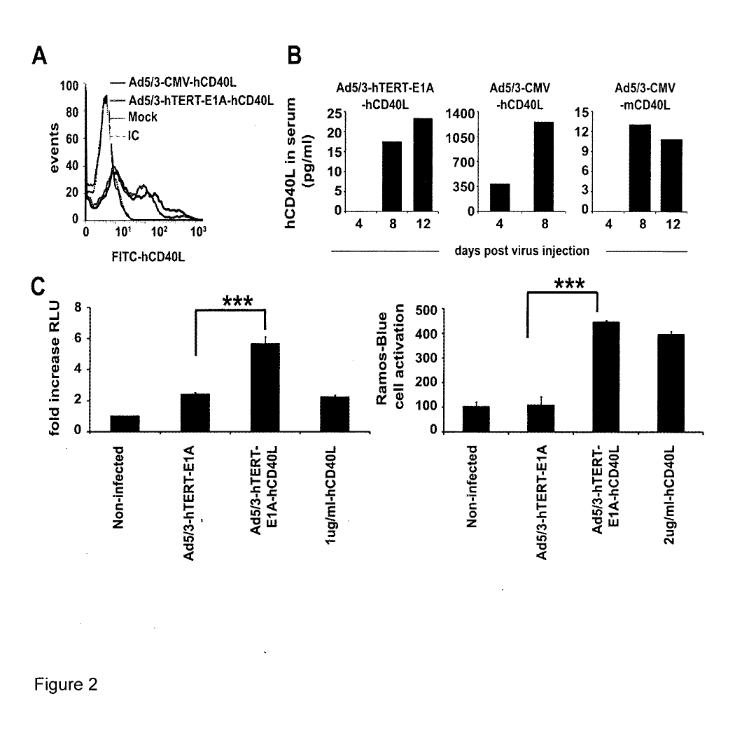

The flow-cytometry results shown in FIG. 2a indicate that both replication competent Ad5/3-hTERT-E1A-hCD40L and replication deficient Ad5/3-CMV-hCD40L resulted in high CD40L expression in vitro.

The expression of hCD40L and mCD40L was confirmed also in vivo with an ELISA analysis (FIG. 2b). Ad5/3-CMV-hCD40L resulted in higher serum levels than Ad5/3-hTERT-E1A-hCD40L as transduced A549 cells are CD40− and not expected to be killed by CD40L. Thus, the transduced cells continue to produce CD40L ad inifinitum, while Ad5/3-hTERT-E1A-hCD40L causes oncolysis of A549 cells which limits the time they have to produce CD40L. This might be advantageous from a safety perspective, as CD40L can cause side effects when present at high concentrations. The human maximum tolerated dose of rhCD40L was reported to correspond with a 2900 pg/ml serum concentration, which is 100-fold higher than what was seen with Ad5/3-hTERT-E1A-hCD40L. Ad5/3-CMV-mCD40L resulted in lower serum mCD40L levels than Ad5/3-CMV-hCD40L, presumably because mCD40L is metabolized by murine tissues and cells, while hCD40L might not be as it is inactive in mice.

The functionality of CD40L expressed by Ad5/3-hTERT-E1A-hCD40L was studied in lung cancer cells (A549). Cell line A549 monolayers (5×10⁶ cells/T25 flask) were infected with 1000 VP/cell of Ad5/3-hTERT-E1A-hCD40L and Ad5/3-hTERT-E1A and one flask was not infected (mock). The supernatant was collected 48 h following the infection and filtered with 0.02 μm filters (Whatman 6809-1002, Maidstone England, England). The supernatant was used for two functionality assays.

In the first functionality assay, EJ cell line monolayers were transfected with the plasmid pNiFty-Luc (InvivoGen) and cultured overnight. pNiFty-Luc is an engineered endothelial cell-leukocyte adhesion molecule (ELAM) promoter combining five NF-κB sites and encoding luciferase. Induction by NF-κB activates the promoter resulting in expression of luciferase.

The supernatant collected from A549 monolayers was added on EJ cells and cultured for 12 hours. One μg/ml recombinant hCD40L protein (Abcam, Cambridge, Mass.) was used as a positive control for the assay. The cells were lysed and the luciferase activity was measured (Luciferase Assay System, Promega, Madison, Wis.). Mock values were subtracted and Nf-κB activity is expressed in fold increase of luciferase expression (relative light units, RLU). The assay was performed three times and each time was assessed in triplicates. Data are presented as mean±SEM; ***, P<0.001 (FIG. 2c).

The second functionality assay was performed on RAMOS-Blue cells. Ramos-Blue cell line is a human B-lymphocyte cell line which stably expresses an NF-κB/AP-1-inducible SEAP reporter gene. When stimulated, these cells produce SEAP in the supernatant which can be measured using the QUANTI-Blue assay reagent (InvivoGen, San Diego, Calif., USA) (FIG. 2d).

These functionality assays show that the virus produces biologically active hCD40L. A 2.3 fold increase is observed in NF-κB activation by hCD40L expressed from replication competent Ad5/3-hTERT-E1A-hCD40L adenovirus in EJ cells transfected with the ELAM plasmid (FIG. 2c) and 4.5 fold increase in NF-κB/AP-1 in Ramos-Blue cells (FIG. 2d). Taken together these results confirm that the constructed viruses express fully functional CD40L both in vitro and in vivo at levels predicted to be safe in humans based on use of recombinant hCD40L.

### Example 3

### Assessment of Oncolytic Potency of the Constructed Adenoviruses In Vitro

For the assessment of the oncolytic potency of the constructed adenoviruses, EJ (CD40+) and A549 (CD40−) cell lines were used. In a cell viability assay, cells on 96-well plates were infected with different concentrations (0.1, 1, 10, 100, 1000 VP/cell) of Ad5/3-hTERT-E1A-hCD40L, Ad5/3-CMV-hCD40L and their control viruses Ad5/3-hTERT-E1A and Ad5/3-Luc1, suspended in a 2% DMEM. One hour later, the cells were washed and incubated in the growth medium containing 5% FCS for 7 days. The cell viability was then analyzed using MTS assay (Cell Titer 96 AQueous One Solution Proliferation Assay, Promega).

For Ad5/3-hTERT-E1A-hCD40L, complete cell killing is seen with 1000 viral particles/cell (VP/cell) in EJ (CD40+) cell line (FIG. 3b). In A549 (CD40−) cell line the oncolytic potency of Ad5/3-hTERT-E1A-hCD40L is slower compared to the control virus Ad5/3-hTERT-E1A (FIG. 3a). When comparing A549 and EJ cell lines infected at the same doses with Ad5/3-hTERT-E1A-hCD40L, a significant increase in cell killing of EJ (CD40+) cells can be seen (FIG. 3c). The same cell lines, when infected with the control replication competent adenovirus Ad5/3-hTERT-E1A, show the opposite results: A549 is more sensitive to the virus (FIG. 3d). These experiments show that Ad5/3-hTERT-E1A-hCD40L has oncolytic potency comparable with the control virus and kills CD40+ cells more efficiently than the positive control virus.

### Example 4

### In Vivo Efficacy of Adenoviruses Expressing hCD40L in Mice

The lack of productive replication of the human adenovirus in mouse cells and the inactivity of hCD40L in mouse tissues complicate preclinical evaluation of Ad5/3-hTERT-E1A-hCD40L. Therefore, it was elected to isolate three antitumor mechanisms into three different mouse models.

For the immune deficient models 10⁶ A549 cells were injected subcutaneously into flanks of nude mice (n=5 mice/group). When tumors reached the size of approximately 5×5 mm, the mice bearing subcutaneous A549 (CD40−) (FIG. 4a) or EJ (CD40+) (FIG. 4b) were injected intratumorally with replication deficient adenovirus Ad5/3-CMV-hCD40L at a dose of 10⁸ VP/tumor on three days (0, 2 and 4) and the tumor growth was followed. Mock animals received only PBS. This experiment shows that CD40L has anti-tumor activity in CD40+ cells (FIG. 4b), whereas no anti-tumor activity can be seen in CD40− cells (FIG. 4a).

For the replication competent models, tumors were injected intratumorally with Ad5/3-hTERT-E1A-hCD40L, Ad5/3-hTERT-E1A and mock at a dose of 10⁸ VP/tumor on three days (0, 2 and 4), and tumor volumes were plotted relative to initial size. Ad5/3-hTERT-E1A-hCD40L was found as potent as the positive control virus Ad5/3-hTERT-E1A both in CD40 negative (FIG. 4c) and CD40 positive tumors (FIG. 4d). This experiment demonstrates the oncolytic potency of Ad5/3-hTERT-E1A-hCD40L but does not take into account the immunological activity of CD40L as hCD40L is not active in mice. This experiment further shows that the oncolytic effect of Ad5/3-hTERT-E1A-hCD40L is not hampered by transgene expression (FIGS. 4c, 4d).

For the immunocompetent model, 5×10⁵ MB49 cells were injected subcutaneously on shaved flanks of C57Black mice (n=7 mice/group). Ad5/3-CMV-mCD40L virus was injected three times intratumorally at the dose of 3×10⁸VP/tumor on days 0, 2 and 4, when tumors reached the size of approximately 5×5 mm. The tumor growth was followed and organs/tumors were collected at the end of the experiments. Tissues were embedded in paraffin and histology and immunohistochemistry (see Example 5 below) were performed.

To study the effect of oncolysis together with the induction of apoptosis by CD40L, without confounding due to immunological effects, a CD40+ xenograft model (nude mice without T-cells) was used. CD40L expression had anti-tumor activity per se (FIG. 4b), and Ad5/3-hTERT-E1A-hCD40L was as effective as the positive control virus showing that the oncolytic effect of Ad5/3-hTERT-E1A-hCD40L is not hampered by transgene expression (FIG. 4d).

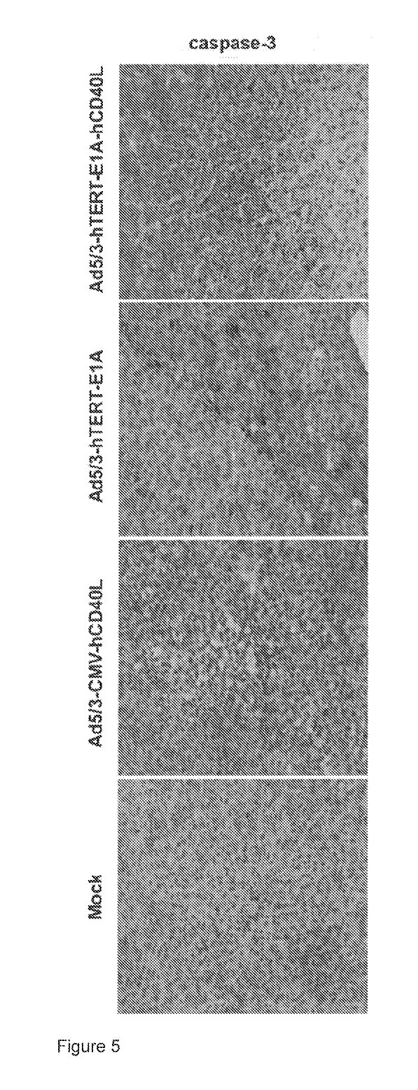

Additionally, to show that CD40L promotes apoptosis in CD40+ tumors in nude mice, mice bearing EJ (CD40+) tumors were injected intratumorally with Ad5/3-Luc1 (mock), Ad5/3-hTERT-E1A, Ad5/3-hTERT-E1A-hCD40L, Ad5/3-CMV-hCD40L three times. After 26 days animals were killed and tumors were collected and embedded in paraffin blocks (n=5mice/group). Immunohistochemistry (for details, see Example 5 below) for caspase-3 was performed to study potential induction of apoptosis. While some apoptosis was induced by Ad5/3-hTERT-E1A as reported for oncolytic adenoviruses and by Ad5/3-CMV-hCD40L due to its hCD40L expression and its apoptotic effect, much more was seen with the Ad5/3-hTERT-E1A-hCD40L (FIG. 5).

### Example 5

### Antitumor Activity of mCD40L in Syngeneic Immunocompetent Animals

Immunohistochemistry used in the analysis of mCD40L in a syngeneic immunocompetent animal model was performed as follows. Tissues were fixed in 4% formalin and paraffin blocks were made. Tissue sections of 4 μm thickness were prepared and incubated with a primary antibody at the dilutions mentioned in Table 1. The sections were incubated with detection kits either for rabbit using LSAB2+ Dako System (DakoCytomation, Carpinteria, Calif., USA (K0673)) or with IHC Select kit (DAB150-RT, Millipore, Mass., USA) for the antibodies raised in rats. Sections were counterstained with hematoxyline and dehydrated in ethanol, clarified in xylene and sealed with Canada balsam. Pictures were taken with an Axioplan2 microscope (Carl Zeiss) equipped with Axiocam (Zeiss).

TABLE 1
Antibodies used in the study

| | Dilution | | |
|---|---|---|---|
| | factor | Cat. No | Company |
| FITC Mouse Anti-human CD40L | 1:5 | 555699 | BD Pharmigen |
| FITC Mouse IgG1 | 1:5 | 555909 | BD Pharmigen |
| Anti human CD40 | 1:100 | VP-C349 | Vector |
| | | | Laboratories |
| Rabbit Anti-active Caspase-3 | 1:100 | 559565 | BD Pharmigen |
| Rabbit Anti-mouse F4/80 | 1:100 | 14-4801 | ebioscience |
| Rat Anti-mouse CD45 | 1:100 | 550539 | BD Pharmigen |
| Rat Anti-mouse CD19 | 1:50 | 14-0193 | ebioscience |
| Rat Anti-mouse CD4 | 1:50 | 14-0041 | ebioscience |
| Rat Anti-mouse CD8 | 1:100 | 14-0083 | ebioscience |

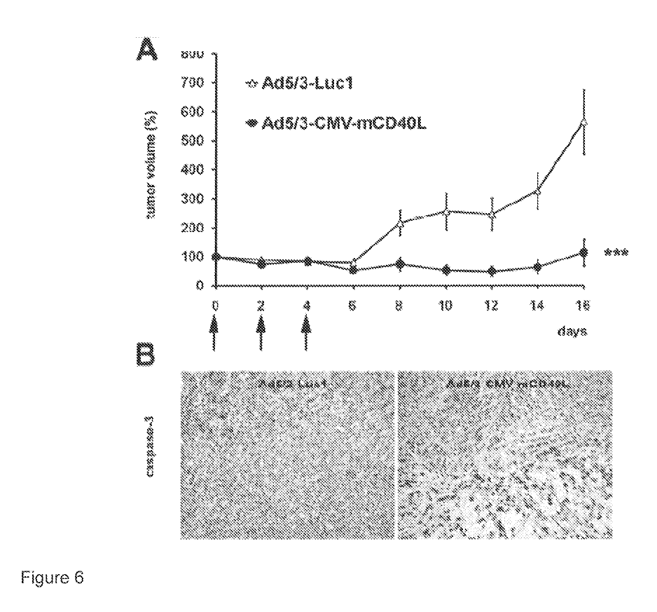

To study the immunological effect of CD40L without confounding due to the effect of oncolysis, C57Black mice bearing subcutaneous MB49 tumors were injected intratumorally with replication deficient adenovirus Ad5/3-CMV-mCD40L or control Ad5/3-Luc1 at a dose of 3×10⁸VP/tumor on days 0, 2 and 4 (n=6 mice/group). Tumor size was followed and plotted relative to the size on day 0. Data are presented in FIG. 6a. There is a significant increase in antitumor activity (p=0.001) in the group treated with Ad5/3-CMV-mCD40L.

For the immunohistochemical analysis of apoptosis (active caspase-3) in tumor treated with Ad5/3-CMV-mCD40L and Ad5/3-Luc1, the tumors were collected 16 days following the first treatment and analyzed for caspase-3 activity. The results are shown in FIG. 6b. Positive staining, i.e. active caspase-3 expression shown in brown, suggests that the anti-tumor activity was partially due to apoptosis induced by binding of mCD40L to CD40+ MB49 cells (FIG. 6b).

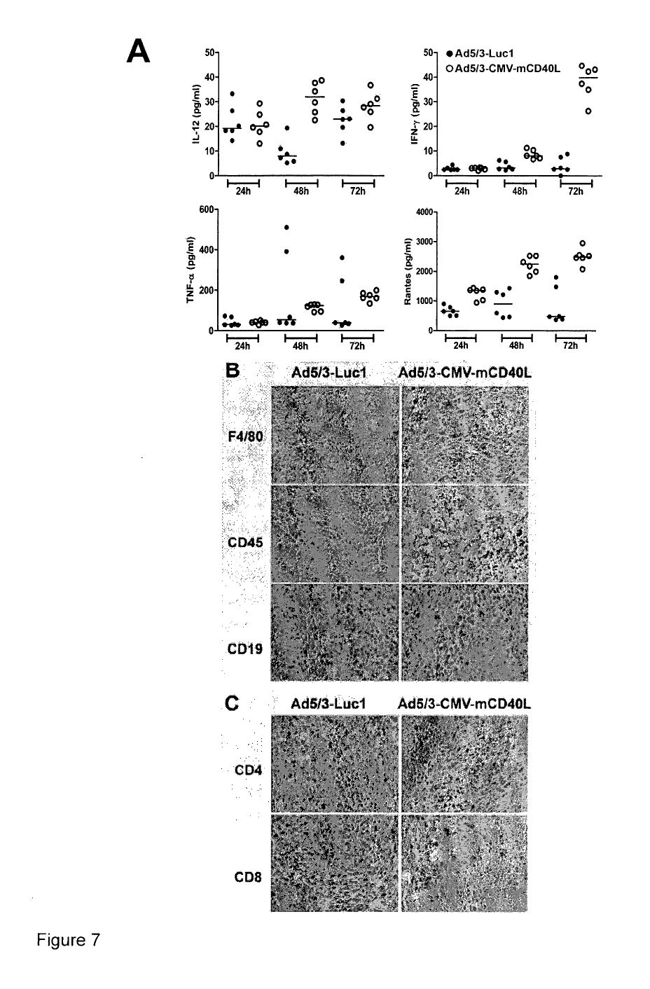

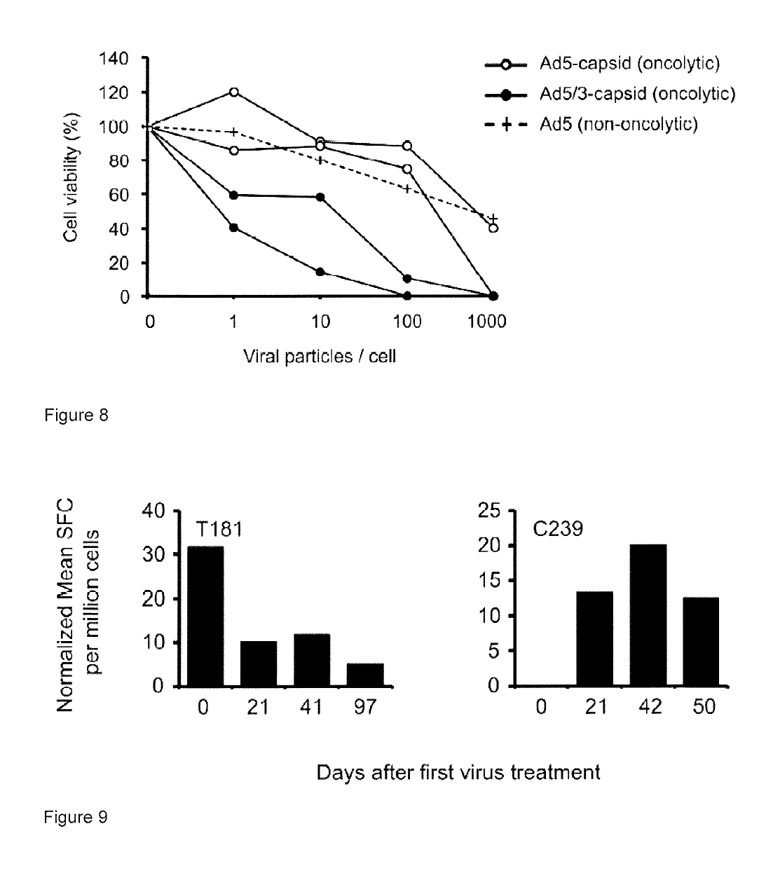

For the analysis of host immune responses in a syngeneic murine model, 4 μm tumor sections were stained by immunohistochemistry for different markers: macrophage (F4/80), leukocytes (CD45) and B-lymphocytes (CD19) (FIG. 7a). Tumor sections were also stained for helper (CD4+) and cytotoxic (CD8+) T cells (brown) (FIG. 7c).

### Example 6

### Effect on Antigen Presenting Cells

An important part of the putative antitumor activity of CD40L coding viruses is their effect on antigen presenting cells. Cytokine analysis for IL-12, TNF-α, INF-γ and RANTES was performed from serum and supernatant of cultured splenocytes from mice treated with Ad5/3Luc1 or Ad5/3-CMV-mCD40L using BD FACSArray according to manufacturer's protocol (BD Cytometric Bead Array Mouse Flex Sets, BD Biosciences). Spleens were minced and splenocytes were cultured in 10% DMEM supplemented with 1% L-glutamine and penicillin/streptomycin. Supernatants were collected at 24, 48, and 72 hours and analyzed for cytokines by FACSArray.

The FACSArray analysis shows increased INF-γ, TNF-α, RANTES and IL-12 production in the group treated with Ad5/3-CMV-mCD40L (FIG. 7A). IL-12 induction suggests the activation of macrophages, an important class of antigen presenting cells. INF-γ, TNF-α, and RANTES are indicators of Th1 type immunity and suggest induction of a cytotoxic T-cell response.

To correlate this on a cellular level, histologic sections of tumors collected 16 days after Ad5/3-CMV-mCD40L injection were analyzed. Enhanced recruitment of macrophages (F4/80) and leukocytes (CD45) was seen, but only a small increase in B-lymphocytes (CD19) was seen, suggesting that the infiltrate was mostly T-cells (FIG. 7b). Analysis of T-cell subsets showed that most of these cells were CD8+ cytotoxic T-cells, although a smaller increase was seen also in CD4+ helper T-cells (FIG. 7c). These findings indicate that production of mCD40L in syngeneic MB49 tumors in immunocompetent animal model prompted a strong anti-tumor immune response mediated through Th1 responsive elements and cytotoxic T-cell infiltration (FIG. 7).

### Example 7

### Pre-Treatment Analysis of Tumor Samples for Prediction of Treatment Efficacy in a Human Patient

For one patient (R73), fresh pretreatment malignant pleural effusion was available for cell killing assay (MTS-assay). Oncolytic adenoviruses with chimeric Ad5/3 capsid (capsid identical with Ad5/3-hTERT-E1A-hCD40L) showed more efficient killing of patient's pleural effusion cells in comparison to oncolytic adenoviruses with serotype 5 capsid. Interestingly, tumor marker Ca15-3 of this patient gradually decreased following the treatment with Ad5/3-hTERT-E1A-hCD40L and a 44% reduction was seen 74 days after treatment (Table 3). Data suggest that adenoviruses with chimeric Ad5/3 capsid efficiently kill human tumor cells and the ex vivo cell killing assay might be interesting for testing of prediction of clinical utility.

### Example 8

### Safety and Efficacy of Ad5/3-hTERT-E1A-hCD40L in Human Cancer Patients

I. Patients

Patients with advanced and treatment refractory solid tumors were enrolled in a Finnish Medicines Agency regulated Advanced Therapy Access Program. Information on patients receiving Ad5/3-hTERT-E1A-hCD40L, doses and prior therapies is listed in Tables 2 and 3.

9 patients, three females (R73, N235, R8) and 6 males (T181, C239, C229, I244, P251, C220), with advanced solid tumors refractory to standard therapies (Table 2) were treated with a single treatment of Ad5/3-hTERT-E1A-hCD40L (R73) or with a serial treatment of Ad5/3-hTERT-E1A-hCD40L (T181, C239, I244, P251, N235, C220) intratumorally, intravenously or intraperitoneally (Table 2). Patient C229 received a serial treatment with Ad5-RGD-D24-GMCSF (PCT/FI2009/051025) and Ad5/3-hTERT-E1A-hCD40L and patient R8 received a serial treatment with Ad5-D24-GMCSF, Ad5/3-hTERT-E1A-hCD40L, and Ad3-hTERT-E1 (WO2010/086838). Inclusion criteria were solid tumors refractory to conventional therapies, WHO performance score 3 or less and no major organ function deficiencies. Exclusion criteria were organ transplant, HIV, severe cardiovascular, metabolic or pulmonary disease or other symptoms, findings or diseases preventing oncolytic virus treatment. Written informed consent was obtained and treatments were administered according to Good Clinical Practice and the Declaration of Helsinki.

TABLE 2
Baseline characteristics and description of treatment

| | | Age | | WHO^{a} | Concomitant | Virus | Virus Dose | Route |
|---|---|---|---|---|---|---|---|---|
| | ID | (Sex) | Tumor type | (ECOG) | Therapies | name | (VP) | (it/iv/ip) |
| Single | R73 | 58 (F) | Breast cancer | 1 | cyclo po | CGTG-401 | 3 × 10e11 | 50/0/50 |
| treat. | | | | | | | | |
| Serial | T181 | 61 (M) | Thyroid cancer | 2 | cyclo po | CGTG-401 | 6 × 10e10 | 100/0/0 |
| treat. | | | | | cyclo po | CGTG-401 | 1 × 10e11 | 100/0/0 |
| | | | | | cyclo po | CGTG-401 | 1 × 10e11 | 100/0/0 |
| | C239 | 56 (M) | Colon cancer | 2 | cyclo po | CGTG-401 | 3 × 10e10 | 100/0/0 |
| | | | | | cyclo po | CGTG-401 | 3 × 10e11 | 100/0/0 |
| | | | | | none | CGTG-401 | 1 × 10e11 | 100/0/0 |
| | C229 | 55 (M) | Colon cancer | 2 | cyclo po | CGTG-XX1 | 5 × 10e11 | 80/20/0 |
| | | | | | cyclo po | CGTG-XX1 | 5 × 10e11 | 100/0/0 |
| | | | | | cyclo po | CGTG-401 | 1 × 10e11 | 100/0/0 |
| | I244 | 49 (M) | Choroideal melanoma | 1 | none | CGTG-401 | 1 × 10e11 | 80/20/0 |
| | | | | | none | CGTG-401 | 1 × 10e11 | 100/0/0 |
| | | | | | none | CGTG-401 | 3 × 10e11 | 100/0/0 |
| | P251 | 62 (M) | Prostate cancer | 0 | cyclo po | CGTG-401 | 3 × 10e11 | 80/20/0 |
| | | | | | none | CGTG-401 | 5 × 10e11 | 100/0/0 |
| | | | | | none | CGTG-401 | 5 × 10e11 | 100/0/0 |
| | N235 | 35 (F) | Adrenal cancer | 1 | none | CGTG-401 | 1 × 10e11 | 100/0/0 |
| | | | | | none | CGTG-401 | 1 × 10e11 | 100/0/0 |
| | | | | | none | CGTG-401 | 1 × 10e11 | 100/0/0 |
| | R8 | 63 (F) | Breast cancer | 1 | cyclo po | CGTG-XX2 | 3 × 10e11 | 0/20/80 |
| | | | | | cyclo po | CGTG-401 | 3 × 10e11 | 0/0/100 |
| | | | | | cyclo po | CGTG-XX3 | 2 × 10e12 | 0/20/80 |
| | C220 | 47 (M) | Colon cancer | 2 | cyclo po | CGTG-401 | 2 × 10e11 | 100/0/0 |
| | | | | | none | CGTG-401 | 3 × 10e11 | 100/0/0 |
| | | | | | none | CGTG-401 | 1 × 10e11 | 100/0/0 |

^{a}Performance status at the time of treatment
cyclo po, Metronomic cyclophosphamide 50 mg/day was given orally
VP, viral particles
it, intaratumorally; iv, intravenously; ip, intraperitoneally
CGTG-XX1, Ad5-RGD-D24-GMCSF
CGTG-XX2, Ad5-D24-GMCSF
CGTG-XX3, Ad3-hTERT-E1 (CGTG-201)

TABLE 3
Prior therapies

| | Age | | |
|---|---|---|---|
| ID | Sex | Tumor type | Prior Therapies |
| R73 | 58 | Breast cancer | Operated; vinorelbine + cyclophosphamide-epirubicin-fluorouracil; radiation + tamoxifen; anastrozole; |
| | F | | docetaxel x8; cyclophosphamide-epirubicin-fluorouracil x2; letrozole |
| T181 | 61 | Thyroid cancer | radioiodine x6; radiation x3; operated x2 |
| | M | | |
| C239 | 56 | Colon cancer | cetuximab + oxaliplatin-fluorouracil-leucovorin (FLOX); irinotecan + fluorouracil + folinic acid x7; cetuximab- |
| | M | | irinotecan + fluorouracil + folinic acid x6; bevacizumab-capecitabine + oxaliplatin; bevacizumab + |
| | | | capecitabine; panitumumab-irinotecan x9; capecitabine |
| C229 | 55 | Colon cancer | operated; capecitabine + oxaliplatin; irinotecan + fluorouracil + folinic acid + bevacizumab; cetuximab + |
| | M | | capecitabine; cetuximab + irinotecan |
| I244 | 49 | Choroideal | radioiodine; liver metastases operated; dacarbazine + becacizumab + interferon; bleomycin + vincristine + |
| | M | melanoma | lomustine + dacarbazine + interferon; cisplatin + etoposide; interferon single |
| P251 | 62 | Prostate cancer | goserelin; intensity modulated radiation therapy (IMRT); docetaxel; mitoxantrone + prednisone |
| | M | | |
| N235 | 36 | Adrenal cancer | operated; doxorubicin + etoposide + cisplatin; streptozocin; mitotane; radiation; capecitabine + |
| | F | | gemcitabine; doxorubicin-eluting beads |
| R8 | 63 | Breast cancer | fluorouracil + epirubicin + cyclophosphamide (FEC) x9; radiation + tamoxifen; docetaxel x8; docetaxel + |
| | F | | capecitabine; paclitaxel; paclitaxel + gemcitabine; vinorelbin; paclitaxel + carboplatin; epirubicin x9 |
| C220 | 47 | Colon cancer | folinic acid + fluorouracil + oxaliplatin (FOLFOX); bevacizumab + irinotecan; cetuximab + irinotecan + |
| | M | | fluorouracil + folinic acid; capecitabine; radiation |

### II. Treatments with Adenoviral Vector Encoding hCD40L

Ad5/3-hTERT-E1A-hCD40L and Ad5-RGD-D24-GM-CSF were produced according to clinical grade and the treatment of patients was initiated.

Two different administration schemas were used. For the first round of serial treatment, 4 patients (C229, I244, P251, R8) received four fifths of the dose intratumorally (or intraperitoneally for patient R8 with peritoneal disease) and one fifth intravenously while another 4 patients (T181, C239, N235, C220) received intratumoral injections only. For later rounds of treatment, all patients were treated intratumorally. Patient R73 received single round of treatment and half of the dose was given intratumorally and half intraperitoneally.

The viral doses appear in Table 2. Doses were chosen based on previous data of the inventors with other oncolytic viruses.

The virus was diluted in sterile saline solution at the time of administration under appropriate conditions. Following virus administration all patients were monitored overnight at the hospital and subsequently for the following 4 weeks as outpatients. Physical assessment and medical history were done at each visit and clinically relevant laboratory values were followed.

Side effects of treatment were recorded and scored according to Common Terminology for Adverse Events v3.0 (CTCAE). Since many cancer patients have symptoms due to disease, pre-existing symptoms were not scored if they did not become worse. However, if the symptom became more severe, e.g. pre-treatment grade 1 changed to grade 2 after treatment, it was scored as grade 2.

Tumor size was assessed by contrast-enhanced computer tomography (CT) scanning. Maximum tumor diameters were obtained. Response Evaluation Criteria in Solid Tumors (RECIST1.1) criteria were applied to overall disease, including injected and non-injected lesions. These criteria are: partial response PR (>30% reduction in the sum of tumor diameters), stable disease SD (no reduction/increase), progressive disease PD (>20% increase). Clear tumor decreases not fulfilling PR were scored as minor responses (MR). Serum tumor markers were also evaluated when elevated at baseline, and the same percentages were used.

Patient serum samples were analyzed for both Th1 type cytokines: interferon-γ (IFN-γ), tumor necrosis factor-α (TNF-α) and interleukin-2 (IL-2), and Th2 cytokines: interleukin-4 (IL-4), interleukin-5 (IL-5) and interleukin 10 (IL-10) with Becton-Dickinson cytokine multiplex bead array system (BD FACSArray; BD Biosciences, San Jose, Calif.) according to the manufacturer's instructions. Samples included baseline, 1 month after virus treatment, or 2 months after virus treatment.

### III Safety of Ad5/3-hTERT-E1A-hCD40L in Cancer Patients

Table 4 summarizes adverse events that were recorded during and after virus treatment. Adverse events were graded according to Common Terminology for Adverse Events v3.0 (CTCAE).

TABLE 4
Adverse events

| | Single treatment | | | | | Serial treatment | | | | |
|---|---|---|---|---|---|---|---|---|---|---|
| | (1 patient) | | | | | (8 patients) | | | | |
| | 1 | 2 | 3 | 4 | 5 | 1 | 2 | 3 | 4 | 5 |
| General | | | | | | | | | | |
| Chills | | | | | | 7 | 1 | | | |
| Dizziness | | | | | | 1 | 1 | | | |
| Dyspnea | | | | | | | 1 | | | |
| Fatigue | | 1 | | | | | 6 | 1 | | |
| Fever | | | | | | 5 | 2 | 1 | | |
| Headache | | | | | | 3 | | | | |
| Lower limb edema | | | | | | 1 | 1 | | | |
| Metabolic or laboratory | | | | | | | | | | |
| ALT increased | | | | | | | 1 | | | |
| AST increased | 1 | | | | | 4 | | 2 | | |
| Hyperbilirubinemia | | | | | | 1 | 1 | | | |
| Hyponatremia | 1 | | | | | 4 | | | | |
| Haematological | | | | | | | | | | |
| Haemoglobin decreased | | | | | | 2 | 4 | | | |
| Leukocytopenia | | | | | | | 4 | | | |
| Lymphocytopenia | | 1 | | | | 1 | 5 | 1 | | |
| Thrombocytopenia | | | | | | 3 | | | | |
| Gastrointestinal | | | | | | | | | | |
| Anorexia | | | | | | 3 | 1 | | | |
| Dysphagia | | | | | | | 1 | | | |
| Nausea | | | | | | 3 | 2 | | | |
| Thirstiness | | | | | | | 1 | | | |
| Musculoskeletal/Soft | | | | | | | | | | |
| tissue | | | | | | | | | | |
| Muscle weakness | | | | | | 1 | 1 | | | |
| Neurology | | | | | | | | | | |
| Neuropathy | | | | | | | 1 | | | |
| Pulmonary/Upper | | | | | | | | | | |
| respiratory | | | | | | | | | | |
| Cough | | | | | | | 1 | | | |
| Edema, larynx | | | | | | 1 | 1 | | | |
| Renal/Genitourinary | | | | | | | | | | |
| Urinary frequency | | | | | | | 1 | | | |
| Pain | | | | | | | | | | |
| Injection site | | 1 | | | | 3 | 1 | | | |
| Shoulder | | | | | | | 1 | | | |
| Stomach | | | | | | 3 | | | | |
| Liver | | | | | | 2 | 2 | | | |
| Pain NOS | | | | | | 1 | 1 | | | |

ALT, alanine aminotransferase; AST, aspartate aminotransferase; NOS, not otherwise specified.
One patient receiving single dose and eight patients receiving repeated doses of Ad5/3-hTERT-E1A-hCD40L were evaluated for adverse events (AE).
No grade 4-5 AEs were seen. AEs are graded according to CTCAE version 3.0.

Ad5/3-hTERT-E1A-hCD40L was well tolerated up to the highest dose used: 5×10¹¹ VP/patient. No grade 4-5 adverse events were seen. The patient treated with a single treatment with Ad5/3-hTERT-E1A-hCD40L experienced only Grade 1 symptoms, whereas the serial treatment patients experienced Grade 1 to 3 symptoms including fatigue, nausea, and transient increase in liver enzymes.

### IV. Neutralizing Antibody Titers

Neutralizing antibodies (NAb) against the Ad5/3 capsid were measured in patients T181 and C239 (Table 5). 293 cells were seeded on 96-well plates at 1×10⁴ cells/well and cultured overnight. Next day, the cells were washed with DMEM without FCS. To inactivate complement, human serum samples were incubated at 56° C. for 90 min. A four-fold dilution series (1:1 to 1:16 384) was prepared in serum-free DMEM (Sarkioja M et al. 2008, Gene Ther 15(12): 921-9). Ad5/3luc1 was mixed with the serum dilutions and incubated at room temperature for 30 min. Next, the cells in triplicates were infected with 100 VP/cell in 50 μl of the above mixture, and 1 h later 100 μl of growth medium with 10% FCS was added. 24 h post-infection, the cells were lysed and luciferase activity was measured with Luciferase Assay System (Promega, Madison, Wis.) utilizing TopCount luminometer (PerkinElmer, Waltham, Mass.). Luciferase readings were plotted relative to gene transfer achieved with Ad5/3luc1 alone in order to evaluate the effect of neutralizing antibodies in the serum of patients treated with the virus.

Data are presented in Table 5 as a serum dilution factor causing 80% inhibition of gene transfer with Ad5/3-luc1 (capsid identical to Ad5/3-hTERT-E1A-hCD40L) to 293 cells. Patient T181 had a high NAb titer already before the treatment and no changes were seen during the treatment. Patient C239 showed initially a low NAb titer. The first treatment induced a high NAb production, which declined to a moderate level along with the treatment. This is not usually seen in patients treated with oncolytic viruses (normally titer increase continuously) and may be an indicator of Th2->Th1 activity.

TABLE 5
Induction of neutralizing antibodies (NAb) and treatment efficacy

| | | NAb titer | | | | TREATMENT RESPONSES | | |
|---|---|---|---|---|---|---|---|---|
| | | Weeks post-treatment^{a} | | | | RECIST^{b} | Tumor | |
| Cohort | Code | 0 | 1-3 | 4-6 | 7- | or PERCIST^{c} | markers^{d} | Survival |
| A | R73 | | | | | na | CR | 154^{e} |
| B | T181 | 4096 | 4096 | 4096 | 4096 | CT: SD (−1%) | PR (−56%) | 195^{e} |
| | C239 | 16 | 4096 | 1024 | 1024 | PET-CT: SMD | PD (+76%) | 126 |
| | | | | | | (−25% SUVpeak) | | |
| | C229 | | | | | CT: PD (+21%) | SD (−8%) | 210^{e} |
| | I244 | | | | | PET-CT: SMD | na | 168^{e} |
| | | | | | | (+1.2% SUVpeak) | | |
| | P251 | | | | | CT: PD (+50%) | PD (+408%) | 118^{e} |
| | N235 | | | | | na | PR (−58%) | 139^{e} |
| | R8 | | | | | CT: SD | MR (−16%) | 147^{e} |
| | C220 | | | | | na | PD | 95 |

Cohort A, single treatment; Cohort B, serial treatment.
^{a}Data are presented as a serum dilution factor causing 80% inhibition of gene transfer with Ad5/3-luc (capsid identical to Ad5/3-hTERT-E1A-hCD40L) to 293 cells.
^{b}Response Evaluation Criteria in Solid Tumors (The longest sum of tumor diameters was used in evaluation): SD, stable disease; PD, progressive disease (>20% increase).
^{c}Modified PET Response Criteria in Solid Tumors: SMD, stable metabolic disease
na, not available (patient not evaluable)
^{d}Evaluation Criteria for Tumor Markers: CR, complete response (<upper limit of normal); PR, partial response (>30% reduction); MR, minor response (12-29% reduction); SD, stable disease; PD, progressive disease (>20% increase).
^{e}alive in the last follow-up at 16^{th} Aug. 2010.

### V. Efficacy Evaluation

Table 5 reports also the efficacy evaluation of Ad5/3-hTERT-E1A-hCD40L according to RECIST criteria for computer tomography (CT) (Therasse P et al. 2000, J Natl Cancer Inst 92, 205-16) or PERCIST criteria (Wahl et al 2009 J Nucl Med 50 Suppl 1:122 S-50S) for positron emission tomography computer tomography (PET-CT). All patients had progressing tumors prior to treatment. Patient T181 had a stable disease (SD), patient C239 had stable metabolic disease (SMD), patient C229 had a progressive disease (PD), patient 1244 had SMD, patient P251 had PD, and patient R8 had SD. In terms of tumor markers assessed for patients, who had elevated markers at baseline, patient R73 had complete response (CR), patients T181 and N235 showed a partial response of −56% and −58%, respectively, patient R8 showed minor response (MR) of −16%, patient C229 had a stable disease, whereas patients C239, P251, and C220 showed a progressive disease in the tumor marker (Table 5). The overall survival of the patients is also shown in Table 5.

Overall, signs of antitumor efficacy were seen in 7/9 patients.

In addition to objective measurements of anti-tumor activity we also saw clinical and/or subjective benefit in several cases. Patient C229 experienced improvement in performance status (WHO 2 before virus treatments and WHO 1 after serial treatment with CGTG-401) and patient 1244 experienced benefit in general symptoms.

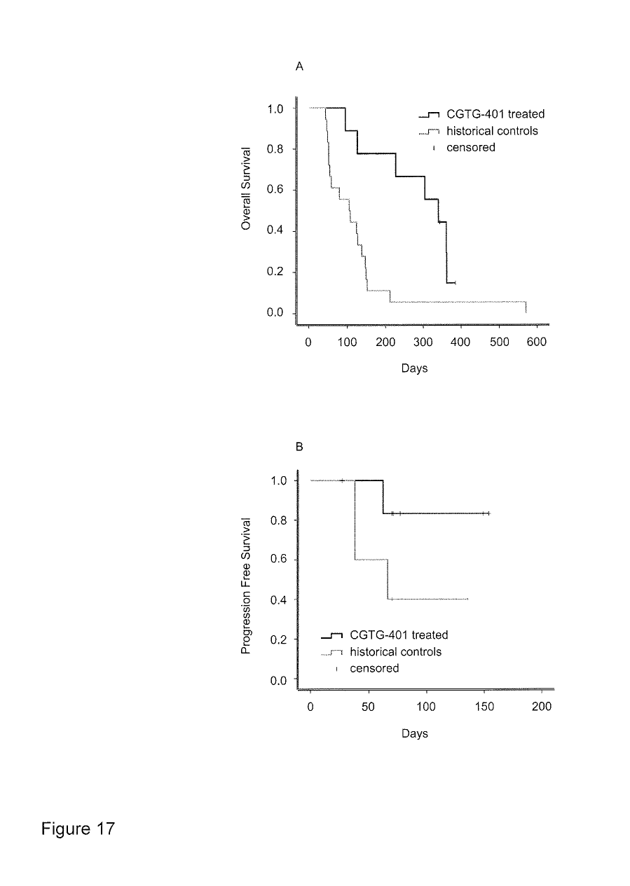

For overall survival analysis of Ad5/3-hTERT-E1A-hCD40L treated patients, cancer patients treated with an unarmed oncolytic adenovirus with an identical capsid (Ad5/3-D24-Cox2L) were used as controls. Survival data were plotted into a Kaplan-Meier curve and cohorts were compared with log-rank test. Median OS for Ad5/3-hTERT-E1A-hCD40L (referred to as CGTG-401 in FIG. 17) and Ad5/3-D24-Cox2L treated patients was 304 and 105 days, respectively, in this non-randomized comparison (p=0.017) (FIG. 17).

### VI. Th1 and Th2 Immune Responses and Inflammation/Toxicity-Related Cytokine Responses after Treatment with Oncolytic Adenovirus Ad5/3-hTERT-E1A-hCD40L

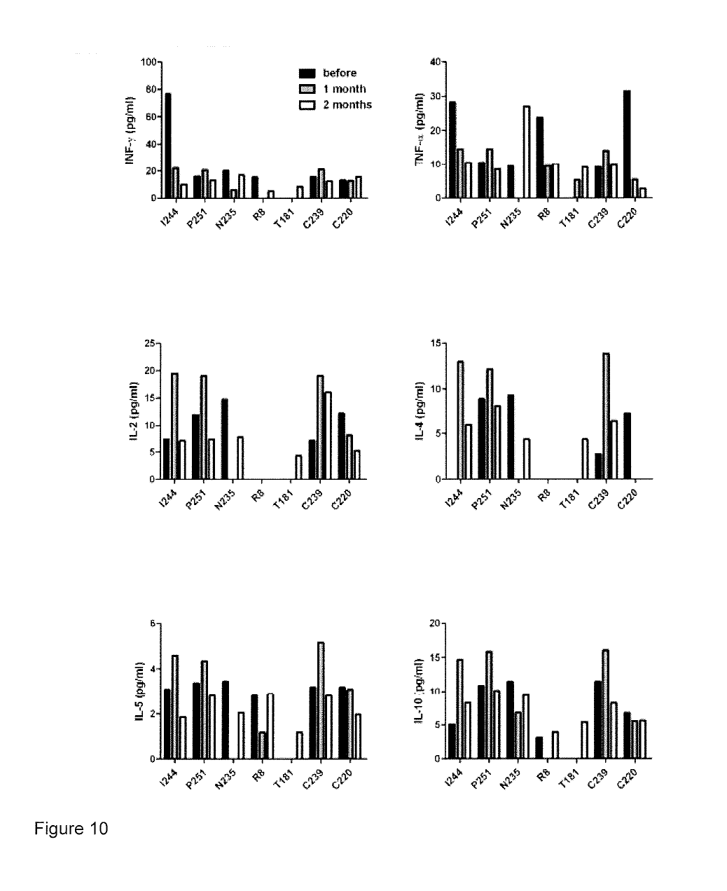

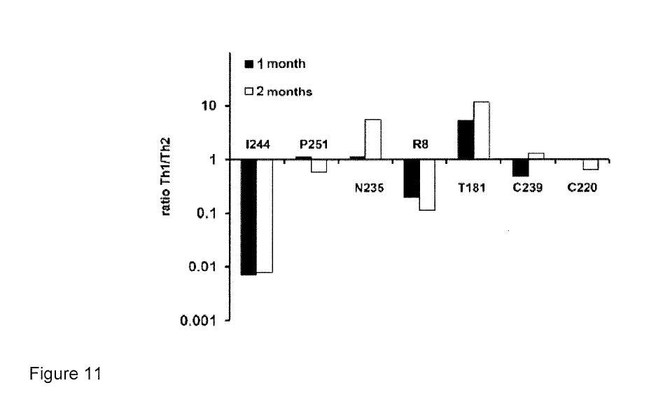

Patient serum samples were analyzed for either Th1 induced cytokines: interferon-γ (IFN-γ), tumor necrosis factor-α (TNF-α) and interleukin-2 (IL-2) or Th2 cytokines: interleukin-4 (IL-4), interleukin-5 (IL-5) and Interleukin 10 (IL-10) with Becton-Dickinson cytokine multiplex bead array system (BD FACSArray; BD Biosciences, San Jose, Calif.) according to the manufacturer's instructions. On the left side of the FIG. 10 are presented Th1 induced cytokines and on the right side the Th2 induced cytokines. Before=serum sample taken before virus was given; 1 month=serum sample taken 1 month after virus treatment; 2 months=serum samples taken 2 months after virus treatment.

In the FIG. 11, cytokine levels are reported relative to their baseline level which was set as 1 and a ratio between Th1/Th2 was calculated for each time point. 1 month=serum sample taken 1 month after virus treatment; 2 months=serum samples taken 2 months after virus treatment. Ratio above 1 indicates dominance of Th1 type immune response whereas ratio below 1 indicates dominance of Th2 type immune response.

Serum levels for interleukin-6 (IL-6) and tumor necrosis factor-alpha (TNF-alpha) were measured to further assess the safety of the treatment. IL-6 and TNF-alpha have been suggested as sensitive markers for acute adenoviral toxicity but no significant increases were seen in these cytokines after treatment (FIG. 13). Furthermore, no post-treatment increases in the serum levels of interleukin-8 (IL-8), interleukin-10 (IL-10), interleukin-12 (IL-12), interferon-γ (IFN-γ) (FIG. 13) were seen.

### VII Induction of Adenovirus and Tumor Antigen Recognizing T-Cells after Treatment with Oncolytic Adenovirus Ad5/3-hTERT-E1A-hCD40L

Oncolytic cell death allows the immune system to gain the capacity for recognizing and killing tumor cells. This is potentially beneficial for tumor eradication and may facilitate cures. Adenovirus is cleared out from the body in a relative short time following the administration; hence it becomes of key importance to stimulate the immune system to be able to recognize specific tumor antigens so that the treatment can result in a sustained beneficial effect for the patient. In addition, when antibodies are induced or present, virus may be partially or fully neutralized so that it can lose its efficacy of infecting and killing metastasis. However, effector T or NK cells induced against the tumor are free to circulate and eventually kill metastasis far from the injected tumor.

In order to demonstrate that the oncolytic adenovirus Ad5/3-hTERT-E1A-hCD40L is able to induce adenovirus recognizing T-cells, total PBMCs were isolated and pulsed with an adenovirus 5 penton-derived peptide pool to assess the activation of adenovirus-specific cytotoxic T-lymphocytes with interferon gamma ELISPOT (FIG. 9). Patient T181 showed decreased number of adenovirus recognizing T-cells following the treatment, whereas patient C239 showed an increase in the number of these cells after treatment. It has been suggested that antiviral immune response may be an important part of the overall antitumor effect mediated by oncolytic viruses (Alemany 2008, Lancet Oncol 9:507-8; Prestwich et al 2008, Lancet Oncol 9:610-2; Tuve et al 2009, Vaccine 27:4225-39).

In addition, for adenovirus ELISPOT, peripheral mononuclear cells (PBMCs) from patients were stimulated with the HAdv-5 Penton peptide pool (Prolmmune, Oxford, UK). For tumor antigen ELISPOT, BIRC5 PONAB peptide, i.e. survivin, was used (Prolmmune). No pre-stimulation or clonal expansion of PBMCs was done in this assay and thus the results indicate the actual frequency of these cells in the blood.

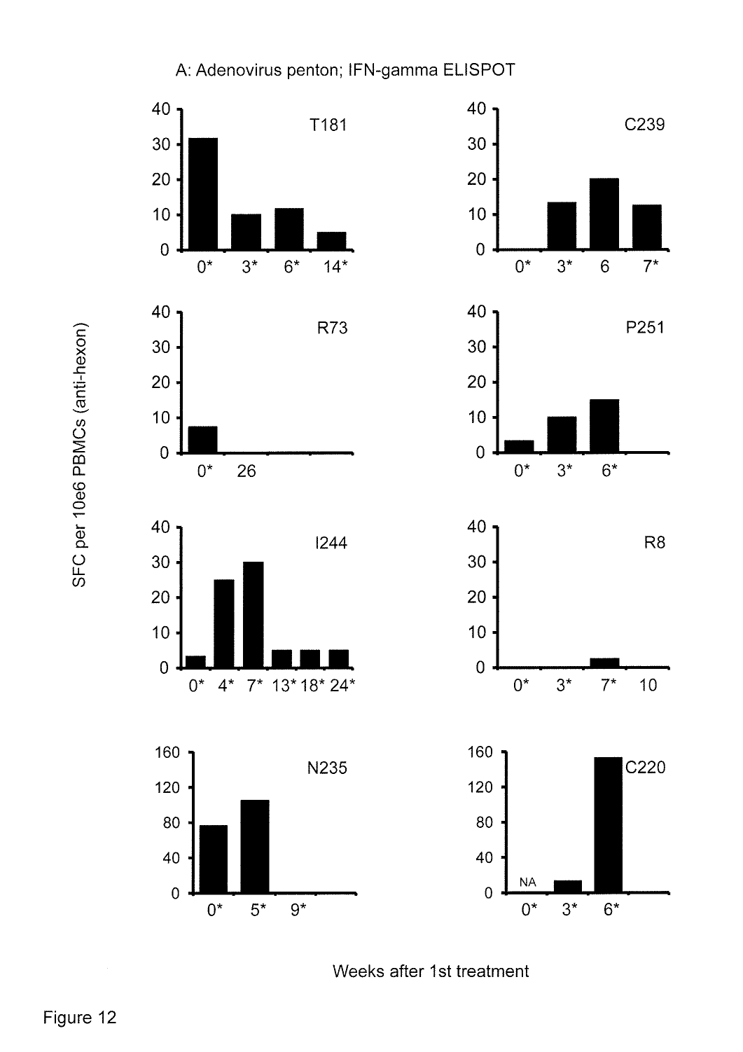

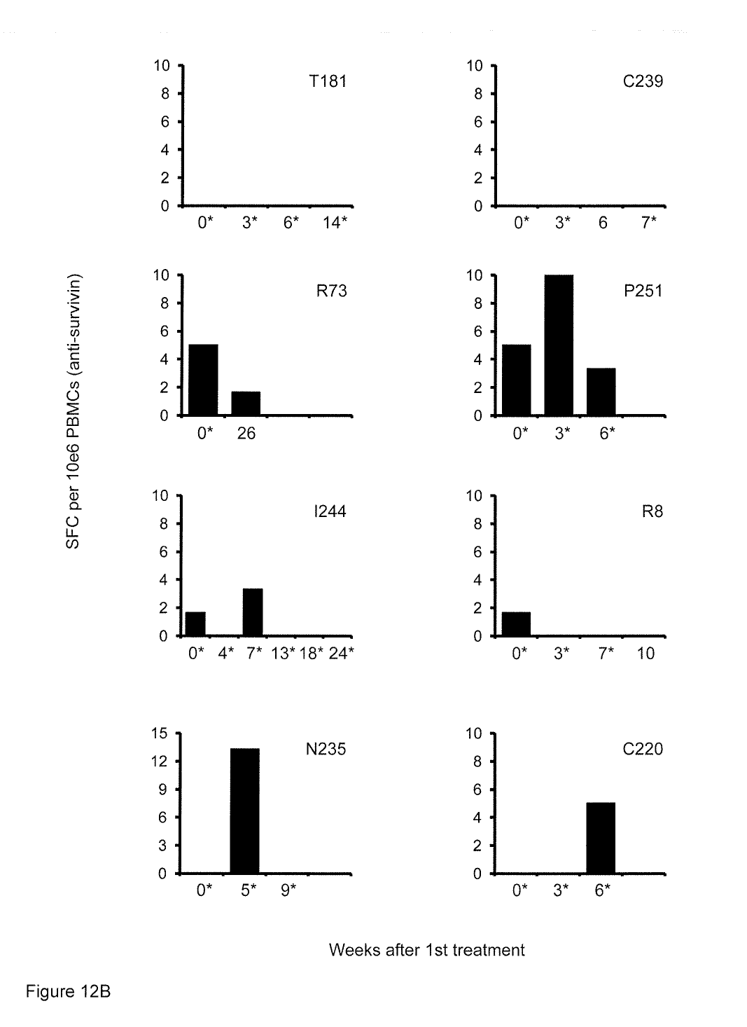

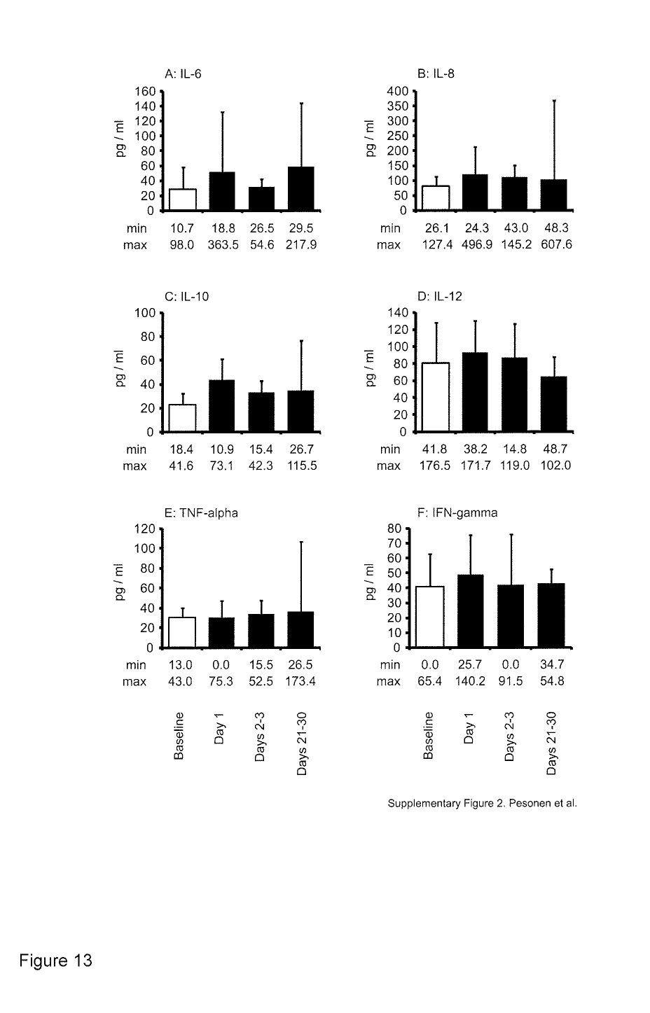

4 out of 8 evaluable patients (C239, P251, I244, C220) showed an increase in systemic adenovirus-specific PBMCs suggesting induction of anti-adenoviral immunity (FIG. 12A). Two patients (T181, R73) showed a decrease, which could be due to trafficking of the cells to the tumor, where viral concentrations are highest. Patient N235 showed a minor increase in adenovirus-specific PBMCs 5 weeks after the virus injection but no circulating adenovirus-specific PBMCs were seen after 9 weeks from the first treatment. R8 showed constantly low or undetectable levels of circulating adenovirus-specific PBMCs.

With regard to survivin-specific response, 4 out of 8 evaluable patients (P251, N235, C220, I244) showed an increased number of PBMCs after treatment (FIG. 12B). In patients R73 and R8 a decrease in the number of tumor-specific T-cells was seen, perhaps suggesting trafficking to the tumor site. Patients T181 and C239 had undetectable levels of circulating surviving-specific PBMCs.

### VIII Analysis of Intracellular Cytokines of Patient PBMCs

Survivin is a useful prototype target for estimating tumor specific immunity since it is expressed by most tumors. However, it is probably not the most immunogenic epitope and therefore anti-survivin T cells could underestimate induction of anti-tumor immunity. Ex vivo expansion of anti-tumor cells, followed by testing of the reactivity against a pool of tumor specific epitopes, could provide an alternative view on anti-tumor immunity, and therefore tumor-specific CD8+ and CD4+ T-cells were measured with intracellular cytokine analysis, when sufficient cell numbers were available.

PBMCs were pulse-stimulated upon thawing with either a hAd5 mixture of hexon and penton peptides or with a mixture of 3-7 TAA PepMixes chosen by cancer type in concentration of 1 μg/mL. After stimulation, cells were fed with CTL growth medium RPMI 1640 (HyClone, Logan, Utah)+Click's Medium (EHAA; Irvine Scientific, Santa Ana, Calif.) 1:1, supplemented with 5 Human AB Serum (Valley Biomedical) and 2 mmol/L L-glutamine (GlutaMAX TM-I; Invitrogen, Carlsbad, Calif.) containing either IL-4 and IL-7 (hAd5-pulsed cells), or IL-12 and IL-7 (TAA-pulsed cells; R&D Systems, Minneapolis, Minn.) in a concentration of 1000 U/mL for IL-4 and in a concentration of 10 ng/mL for IL-7 and IL-12. After 10 days in culture, the cells were re-stimulated with hAd5 or TAA peptide mixes as previously with CD28 and CD49 (0.1 μg/ml; BD, Franklin Lakes, N.J., USA) added for co-stimulation, and surface stained with monoclonal antibodies to CD3 and CD8 (Becton Dickinson, Franklin Lakes, N.J.) in saturating amounts (5 μl). Cells were stained for cytokines with 20 μl FITC-anti-IFN-γ or Pe-anti-TNF-α-antibody (BD Biosciences) and analyzed using a FACSCalibur equipped with Cell Quest software (BD, San Diego, Calif.).

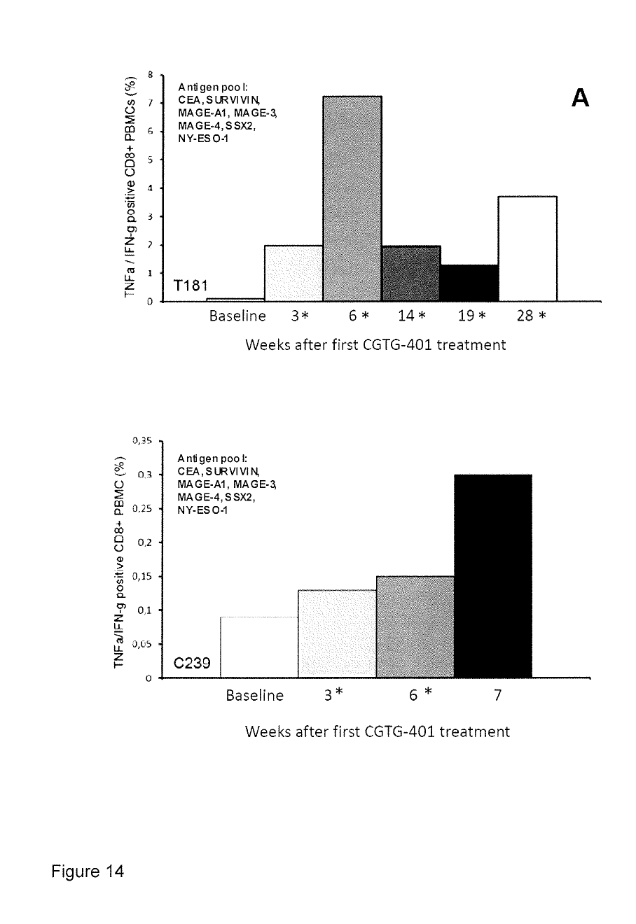

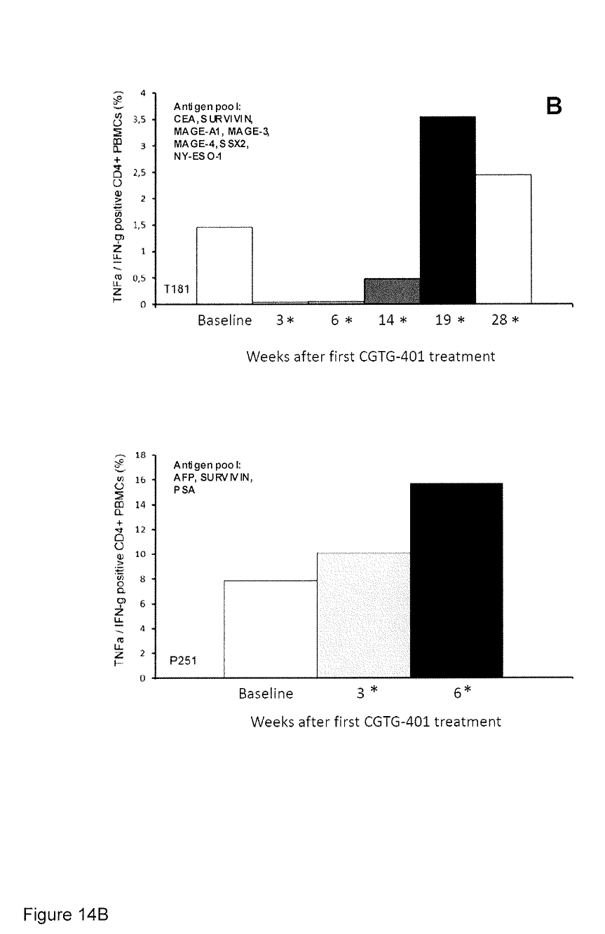

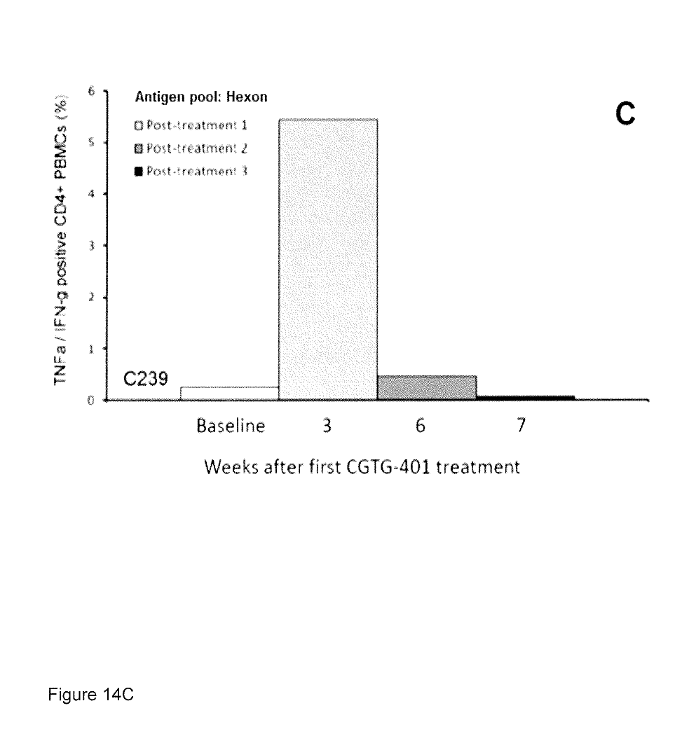

Patients T181 and C239 showed an increase in tumor-specific CD8+ T-cells in all post-treatment measurements (FIG. 14A). With regard to CD4+ cells, T181 and P251 showed an increase in comparison to baseline (FIG. 14B). Furthermore, the number of adenovirus recognizing CD4+ T-cells were studied for patient C239 and a transient increase was seen three weeks after virus administration (FIG. 14C), confirming ELISPOT data (see item VII).

### IX. Analysis of Local and Systemic Levels of CD40L and RANTES

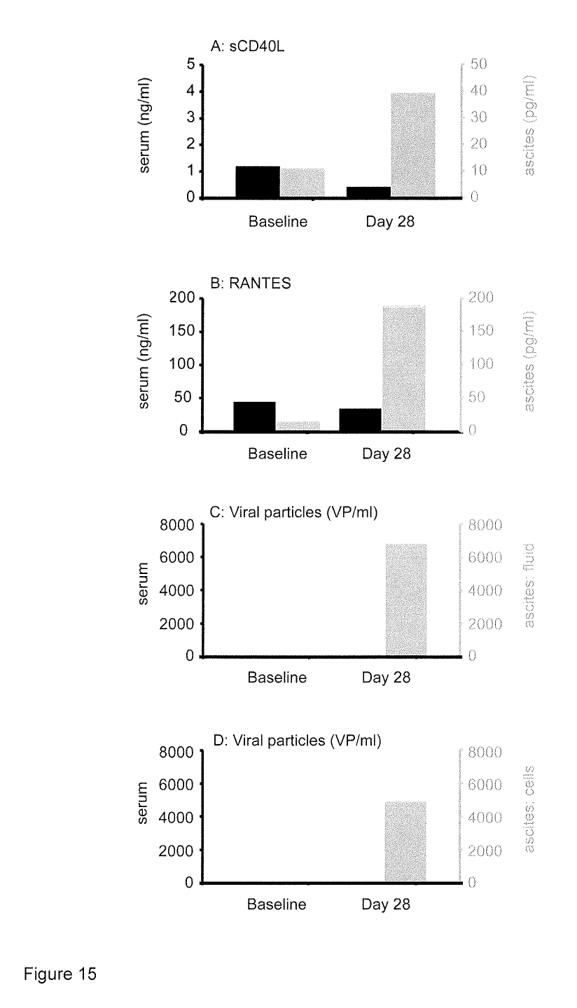

In order to evaluate local levels of CD40L and RANTES (a down-stream molecule whose expression is determined in part by CD40L) in malignant ascites soluble CD40L (sCD40L) and RANTES concentrations in the fluid were assessed and compared to systemic levels of these cytokines (FIG. 15A and FIG. 15B, respectively). Malignant ascites (resulting from peritoneal tumor masses) was removed from the peritoneal cavity of patient R8 before and 28 days after virus administration and the CD40L and RANTES concentrations were analyzed using BD Cytometric Bead Array (CBA) Human Soluble Protein Flex Set (BD, San Diego, Calif.). Both sCD40L and RANTES levels increased locally at the tumor whereas no increases in systemic levels were seen.

Additionally, the amount of viral particles (VP) in ascites fluid (FIG. 15C) and in cells isolated from ascites (FIG. 15D) was analyzed to assess the replication of the virus at the tumor site. The analysis was done by qPCR using primers and probe targeting the E3 region flanking the CD40L sequence (forward primer 5″-CCGAGCTCAGCTACTCCATC-3′, SEQ ID NO: 6, reverse primer 5″-GCAAAAAGTGCTGACCCAAT -3′, SEQ ID NO: 7 and probe onco 5′FAM-CCTGCCGGGAACGTACGATG-3′MGBNFQ, SEQ ID NO: 8, SEQ ID NO: 9, SEQ ID NO: 10). High amounts of virus suggestive of replication in the tumor were found in ascites fluids and cells on day 28 after virus treatment, whereas no virus was detected in the serum on the same day.

Thus, both sCD40L and RANTES levels increased locally at the tumor whereas no increases in systemic levels were seen, suggesting that immunological effects were restricted to the tumor site.

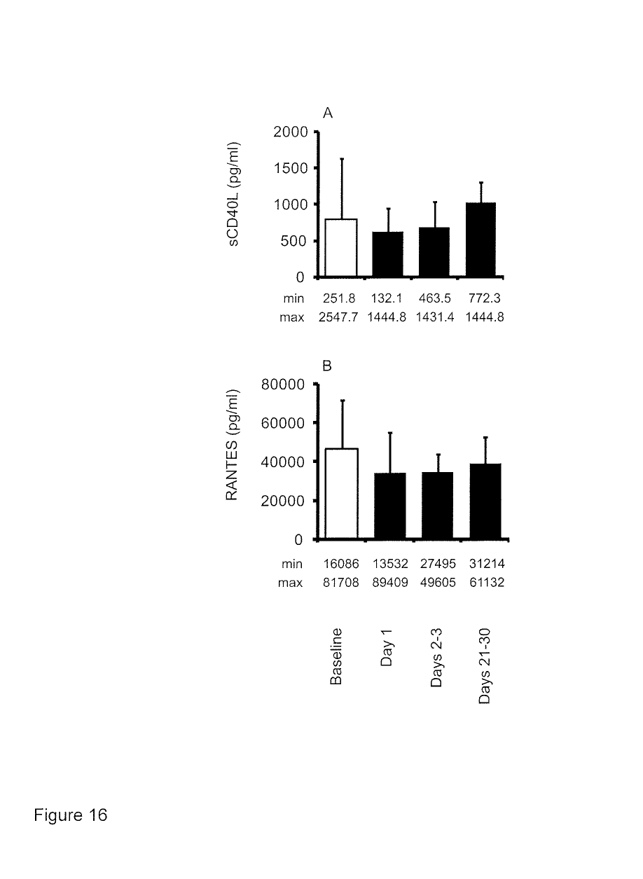

To further confirm that the immunological effects are locally restricted, systemic levels for sCD40L and RANTES for all 9 patients were assessed before and after virus treatment. No significant increases were seen in any patients, suggesting that immunological effects were restricted to the tumor site as predicted by virus design (FIG. 16). Blood samples for all 9 patients were collected before (baseline) and at several time points after the treatment. Soluble CD40L (sCD40L) and RANTES concentrations in the serum were assessed. Data are presented as median±SD.

1.-33. (canceled)

34. An oncolytic adenoviral vector comprising
1) an adenovirus serotype 5 (Ad5) nucleic acid backbone comprising a capsid modification,
2) a nucleic acid sequence encoding a tumor specific human telomerase reverse transcriptase (hTERT) promotor upstream of the E1 region; and
3) a nucleic acid sequence encoding human CD40L in the place of the deleted adenoviral genes gp19k/6.7K in the E3 region.

35. An oncolytic adenoviral vector according to claim 34 further comprising one or more regions selected from a group consisting of E2, E4, and late regions.

36. An oncolytic adenoviral vector according to claim 34, wherein a wild type region is located upstream of the E1 region.

37. An oncolytic adenoviral vector according to claim 34, wherein the E1 region comprises a viral packaging signal.

38. An oncolytic adenoviral vector according to claim 34, wherein a nucleic acid sequence encoding CD40L is under the control of the viral E3 promoter.

39. An oncolytic adenoviral vector according to claim 34, wherein a nucleic acid sequence encoding CD40L is one nucleotide distinct from the wild type human sequence.

40. An oncolytic adenoviral vector according to claim 34, wherein the E4 region is of a wild type.

41. An oncolytic adenoviral vector according to claim 34, wherein the capsid modification is Ad5/3 chimerism, insertion of an integrin binding (RGD) region and/or heparin sulphate binding polylysine modification into the fiber.

42. An oncolytic adenoviral vector according to claim 41, wherein the capsid modification is a RGD-4C modification.

43. A cell comprising the adenoviral vector according to claim 34.

44. A pharmaceutical composition comprising the adenoviral vector according to claim 34.

45. An oncolytic adenoviral vector or pharmaceutical composition according to claim 34, which acts as an in situ cancer vaccine.

46. Adenoviral vector according to claim 34 for treating cancer in a subject.

47. A method of treating cancer in a subject, wherein the method comprises administration of the vector or pharmaceutical composition according to claim 34 to a subject.

48. The adenoviral vector or method according to claim 46, wherein the cancer is selected from a group consisting of nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, oligodendroglioma, neuroblastoma, meningioma, spinal cord tumor, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, esophagus cancer, gall bladder cancer, head cancer, eye cancer, neck cancer, kidney cancer, Wilms' tumor, Kaposi's sarcoma, prostate cancer, testicular cancer, Hodgkin's disease, non-Hodgkin's lymphoma, skin cancer, mesothelioma, multiple myeloma, ovarian cancer, endocrine pancreatic cancer, glucagonoma, pancreatic cancer, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer, and tonsil cancer.

49. The adenoviral vector or method according to claim 46, wherein the subject is a human or an animal.

50. The adenoviral vector or method according to claim 46, wherein the administration is conducted through an intratumoral, intramuscular, intra-arterial, intravenous, intrapleural, intravesicular, intracavitary or peritoneal injection, or an oral administration.

51. The adenoviral vector or method according to claim 46, wherein oncolytic adenoviral vectors or pharmaceutical compositions are administered several times during the treatment period.

52. The adenoviral vector or method according to claim 46, wherein the oncolytic adenoviral vector having a different fiber knob of the capsid compared to the vector of the earlier treatment, is administered to a subject.

53. The adenoviral vector or method according to claim 46, wherein the method further comprises administration of concurrent radiotherapy or concurrent chemotherapy to a subject.

54. The adenoviral vector or method according to claim 46, wherein the method further comprises administration of an auxiliary agent, selected from the group consisting of verapamil or another calcium channel blocker; an autophagy inducing agent; temozolomide; a substance capable to downregulating regulatory T-cells; cyclophosphamide; and any combination thereof in a subject to a subject.

55. The adenoviral vector or method according to claim 46, wherein the method further comprises administration of chemotherapy or anti-CD20 therapy or other approaches for blocking of neutralizing antibodies.

56. A method of producing CD40L in a cell, wherein the method comprises:
a) carrying a vehicle comprising an oncolytic adenoviral vector according to claim 34 to a cell, and
b) expressing CD40L of said vector in the cell.

57. A method of increasing tumor specific immune response in a subject, wherein the method comprises:
a) carrying a vehicle comprising an oncolytic adenoviral vector according to claim 34 to a target cell or tissue,
b) expressing CD40L of said vector in the cell, and
c) increasing amount or activity of cytotoxic T cells and/or natural killer cells in said target cell or tissue.

58. A use of the oncolytic adenoviral vector according to claim 34 for producing CD40L in a cell.

59. A use of the oncolytic adenoviral vector according to claim 34 for increasing tumor specific immune response, Th1->Th2 switch or reduction of immunosuppression in a subject.

60. A use of the oncolytic adenoviral vector according to claim 59, wherein amount of natural killer and/or cytotoxic T cells is increased in a target cell or tissue or suppressor cells (such as regulatory T-cells) are decreased.


SEQUENCE LISTING (10 sequences)

SEQ ID NO: 1 - DNA, 255 bp, Homo sapiens
ctgcgctgtcggggccaggccgggctcccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcag

SEQ ID NO: 2 - DNA, 770 bp, Homo sapiens
  misc_feature (148)..(148): Point mutation in hCD40L
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttgg

SEQ ID NO: 3 - DNA, 24 bp, Artificial Sequence
  primer
tttaacatctctccctctgtgatt

SEQ ID NO: 4 - DNA, 23 bp, Artificial Sequence
  primer
tataaatggagcttgactcgaag

SEQ ID NO: 5 - DNA, 35989 bp, Artificial Sequence
  virus sequence
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactggtaccgcggccgcagaacatttctctatcgataggtaccgctgcgctgtcggggccaggccgggctcccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccgggctcgagctagaagcttgttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagactnnngttttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggatggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaaccctatggacaggtccaaaaccagaagccaactgcataattgaatacgggaaacaaaacccagatagcaaactaactttaatccttgtaaaaaatggaggaattgttaatggatatgtaacgctaatgggagcctcagactacgttaacaccttatttaaaaacaaaaatgtctccattaatgtagaactatactttgatgccactggtcatatattaccagactcatcttctcttaaaacagatctagaactaaaatacaagcaaaccgctgactttagtgcaagaggttttatgccaagtactacagcgtatccatttgtccttcctaatgcgggaacacataatgaaaattatatttttggtcaatgctactacaaagcaagcgatggtgccctttttccgttggaagttactgttatgcttaataaacgcctgccagatagtcgcacatcctatgttatgacttttttatggtccttgaatgctggtctagctccagaaactactcaggcaaccctcataacctccccatttaccttttcctatattagagaagatgactaataaactctaaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcaagtgatattaataaactccccgggcagctcacttaagttcatgtcgctgtccagctgctgagccacaggctgctgtccaacttgcggttgcttaacgggcggcgaaggagaagtccacgcctacatggggggagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgcttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgccccgccgggntatacactgcagggaaccgggacttggacaatgacaagtgggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatatttaagtccgggccattgtaaaaaatttggctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcagtccggagtcataatgtaagactcggtaaacacatcaggttgattcacatcggtcagtgttaaaaagcgaccgaaatagccngggggaatacaatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacggtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 6 - DNA, 20 bp, Artificial Sequence
  forward primer 2
ccgagctcagctactccatc

SEQ ID NO: 7 - DNA, 20 bp, Artificial Sequence
  reverse primer 2
gcaaaaagtgctgacccaat

SEQ ID NO: 8 - protein, 3 aa, Artificial Sequence
  probe_protein part 1
FAM

SEQ ID NO: 9 - DNA, 20 bp, Artificial Sequence
  probe_DNA part
cctgccgggaacgtacgatg

SEQ ID NO: 10 - protein, 6 aa, Artificial Sequence
  probe_protein part 2
MGBNFQ